SEQUENCE LISTING (19 sequences)

SEQ ID NO: 1 - protein, 7 aa, Simian virus 40
PKKKRKV

SEQ ID NO: 2 - protein, 10 aa, Simian virus 40
PKKKRKVEAS

SEQ ID NO: 3 - protein, 16 aa, Simian virus 40
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Homo sapiens
NQSSNFGPMKGGNFGGRSSGPYGGGGQYFAKPRNQGGY

SEQ ID NO: 7 - protein, 42 aa, Homo sapiens
RMRIZFKNKGKDTAELRRRRVEVSVELRKAKKDEQILKRRNV

SEQ ID NO: 8 - protein, 8 aa, Homo sapiens
VSRKRPRP

SEQ ID NO: 9 - protein, 8 aa, Homo sapiens
PPKKARED

SEQ ID NO: 10 - protein, 8 aa, Homo sapiens
PQPKKKPL

SEQ ID NO: 11 - protein, 12 aa, Mus sp.
SALIKKKKKMAP

SEQ ID NO: 12 - protein, 5 aa, Influenza virus
DRLRR

SEQ ID NO: 13 - protein, 7 aa, Influenza virus
PKQKKRK

SEQ ID NO: 14 - protein, 10 aa, Hepatitis D virus
RKLKKKIKKL

SEQ ID NO: 15 - protein, 10 aa, Mus sp.
REKKKFLKRR

SEQ ID NO: 16 - protein, 20 aa, Homo sp.
KRKGDEVDGVDEVAKKKSKK

SEQ ID NO: 17 - protein, 17 aa, Homo sapiens
RKCLQAGMNLEARKTKK

SEQ ID NO: 18 - protein, 288 aa, synthetic construct
  source 1..288: note = N-terminal capping region
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, synthetic construct
  source 1..183: note = C-terminal capping region
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2025/0009805 A1
(43) Pub. Date: Jan. 9, 2025

(54) OLFACTORY EXTRACELLULAR VESICLES AND USES THEREOF

(71) Applicant: CORNELL UNIVERSITY, Ithaca, NY (US)

(72) Inventor: David Lin, Ithach, NY (US)

(21) Appl. No.: 18/708,322

(22) PCT Filed: Nov. 8, 2022
(86) PCT No.: PCT/US2022/079483

§ 371 Date: May 8, 2024

Related U.S. Application Data
(60) Provisional application No. 63/277,038, filed on Nov. 8, 2021.

(51) Int. Cl. A61K 35/30; A61P 25/00; C12N 5/0793

(52) U.S. Cl. CPC A61K 35/30; A61P 25/00; C12N 5/062

(57) ABSTRACT

Described in several embodiments herein are olfactory sensory neuron (OSN) derived extracellular vehicles (EVs), formulations thereof, and uses thereof. In some embodiments, the OSN derived extracellular vesicles are effective to increase the growth of neurons during regeneration. In some embodiments, the OSN derived EVs and/or formulations thereof are used to promote nerve regeneration, particularly peripheral nerve regeneration, and to treat nerve injury.

### CROSS-REFERENCE TO RELATED APPLICATIONS

This application claims priority to and the benefit of U.S. Provisional Patent Application No. 63/277,038 filed Nov. 8, 2021, the contents of the above-identified application are hereby fully incorporated herein by reference in their entirety.

### SEQUENCE LISTING

This application contains a sequence listing filed in electronic form as an .xml file entitled CORNL-0725WP_ST26.xml, created on Nov. 8, 2022 and having a size of 18,268. The content of the sequence listing is incorporated herein in its entirety.

### TECHNICAL FIELD

The subject matter disclosed herein is generally directed to extracellular vesicles and uses thereof.

### BACKGROUND

Peripheral neuropathy involves damage to neurons in the peripheral nervous system (PNS), and has numerous causes, including: traumatic injury, infection, metabolic disorder, exposure to environmental toxins, genetic causes, and others. For example, injury can be caused by damage during surgery, vehicular collision, or prolonged immobility. Recovery time depends on the extent of damage, and regeneration can be as slow as 1-2 mm per day. Critically, there is no treatment to accelerate this regrowth (Pfister B J, Gordon T, Loverde J R, et al. Biomedical engineering strategies for peripheral nerve repair: Surgical applications, state of the art, and future challenges. Critical Reviews in Biomedical Engineering. 2011; 39(2):81-124 and Takahashi K., Yamanaka S. Induction of pluripotent stem cells from mouse embryonic and adult fibroblast cultures by defined factors. Cell. 2006; 126:663-676).

As such there exists an urgent and unmet need for treatments effective for regeneration of damaged neurons, particularly for PNS neurons.

Citation or identification of any document in this application is not an admission that such a document is available as prior art to the present invention.

### SUMMARY

Described in certain example embodiments herein are compositions comprising a population of olfactory sensory neuron (OSN) derived extracellular vesicles (EVs) enriched for EVs comprising and optionally expressing one or more cell adhesion molecules, wherein the one or more cell adhesion molecules are optionally one or more cadherins, one or more neural cell adhesion molecules (NCAMs), one or more epithelial cell adhesion molecule (EpCAM), one or more integrins, one or more plexins, or any combination thereof.

In certain example embodiments, the one or more cell adhesion molecules is/are, a delta protocadherin, optionally Pcdh1, Pcdh7, Pcdh8, Pcdh9, Pcdh10, Pcdh11, Pcdh17, Pcdh18, Pcdh19, Pcdh20 or any combination thereof.

In certain example embodiments, the OSN derived EVs are microvesicles, exosomes, or a heterogenous population of both.

In certain example embodiments, the OSN derived EVs comprise an exogenous cell adhesion molecule gene and/or gene product, wherein the exogenous cell adhesion molecule gene or gene product is optionally one or more cadherins, one or more neural cell adhesion molecules (NCAMs), one or more epithelial cell adhesion molecule (EpCAM), one or more integrins, one or more plexins, or any combination thereof.

In certain example embodiments, the OSN derived EVs comprise an exogenous Pcdh1, Pcdh7, Pcdh8, Pcdh9, Pcdh10, Pcdh11, Pcdh17, Pcdh18, Pcdh19, or Pcdh20 gene or gene product, or any combination thereof.

In certain example embodiments, the composition further comprises a cargo, wherein the cargo is optionally a polynucleotide, a polypeptide, a nutrient (e.g., lipid, amino acid, carbohydrate, peptide, protein, sugar, vitamin, mineral, and/or the like), a genetic modifying system or component thereof, antibody or fragment thereof, aptamer, affibody, small molecule chemical agent (e.g., a therapeutic and/or prevention), an immunomodulator, a hormone, an antipyretic, an anxiolytic, an antipsychotic, an analgesic, an antispasmodic, an anti-epileptic agent, an anti-inflammatory agent, an anti-histamine, an anti-infective, a radiation sensitizer, a chemotherapeutic, a growth factor, a neurotransmitter agonist, a neurotransmitter antagonist, or any combination thereof.

In certain example embodiments, the OSN derived EVs comprise one or more targeting moieties, wherein the targeting moiety is optionally a peptide, polypeptide, polynucleotide, sugar, a chemical molecule, a polymer, a lipid, a glycan, a peptidoglycan, or any combination or complex thereof (e.g., receptors, receptor ligands, antibodies and fragments thereof, aptamers, affibodies, antibody and/or aptamer epitopes, binding agents and their binding partners (e.g., biotin and streptavidin, enzymes and their substrates, a targeting nucleic acid, target nucleic acid and guided nuclease (e.g., miRNA, gRNA, RISC, Cas, etc.), guide nucleic acid for a guided nuclease system, and/or the like.

In certain example embodiments, the composition is frozen, dehydrated, lyophilized, or otherwise modified for storage.

In certain example embodiments, the composition is effective to stimulate axonal growth and/or increase the rate of axonal growth in a peripheral neuron, a central nervous system neuron, or both.

In certain example embodiments, the composition is effective to increase or enhance correct axonal connectivity during neuron regeneration.

Described in certain example embodiments herein are formulations, such as pharmaceutical formulations, comprising a composition of any one of the preceding paragraphs and as further described in greater detail elsewhere herein and a pharmaceutically acceptable carrier or excipient.

In certain example embodiments, the formulation is adapted for oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, buccal, conjunctival, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradiscal, intraductal, intraduodenal, intradural, intraepidermal, intraesophageal, intragastric, intragingival, intraileal, intralesional, intraluminal, intralymphatic, intramedullary, intrameningeal, intraovarian, intrapericardial, intrapleural, intraprostatic, intrapulmonary, intrasinal, intraspinal, intrasynovial, intratendinous, intratesticular, subarachnoid, subconjunctival, subcutaneous, sublingual, submucosal, topical, transdermal, transmucosal, transplacental, transtracheal, transtympanic, or any combination thereof administration.

Described in certain example embodiments herein are methods of treating a disease, disorder, and/or condition in a subject in need thereof, the method comprising administering a composition or a formulation as in any one of the preceding paragraphs or as described in greater detail elsewhere herein to the subject in need thereof.

Described in certain example embodiments herein are methods of increasing/enhancing axonal growth and/or the rate of axonal growth during neuron development and/or regeneration, the method comprising administering a composition or a formulation as in any one of the paragraphs or as described in greater detail elsewhere herein to the subject in need thereof.

Described in certain example embodiments herein are methods of increasing neuron synapse formation, connectivity, or both during neuron development and/or regeneration, the method comprising administering a composition or a formulation as in any one of the preceding paragraphs or as described elsewhere herein to the subject in need thereof.

In certain example embodiments, the subject in need thereof has a nerve injury, nerve death, aberrant neuron connectivity, aberrant neuron activity, a neuropathy, or any combination thereof.

In certain example embodiments, the subject in need thereof has or is suspected of having a neurodegenerative disease, disorder, and/or condition.

In certain example embodiments, the subject in need thereof has, has had, or is suspected of having an epilepsy, a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof.

In certain example embodiments, the subject in need thereof has, has had, or is suspected of having a CNS neuron/nerve and/or a peripheral neuron/nerve injury, disease, disorder, and/or condition.

In certain example embodiments, the disease, disorder, and/or condition is a genetic disease, disorder, and/or condition.

In certain example embodiments, the disease, disorder, and/or condition is not a genetic disease, disorder, and/or condition.

Described in certain example embodiments herein are methods of promoting stem cell division or differentiation and/or cell reprogramming, comprising: administering a composition or a formulation as in any one of the preceding claims to a stem cell or epithelial cell or population thereof.

In certain example embodiments, the cell is a differentiated cell.

In certain example embodiments, the cell is an epithelial cell.

In certain example embodiments, the cell is a neuron cell.

In certain example embodiments, the stem cell is an induced pluripotent stem cell.

Described in certain example embodiments herein are devices comprising a population of olfactory sensory neuron (OSN) derived extracellular vesicles (EVs), wherein the population of OSN derived EVs are fixed in a pattern on one or more surfaces on the device.

In certain example embodiments, the population of OSN derived EVs are dried.

In certain example embodiments, the pattern is configured to direct correct neuron growth.

In certain example embodiments, the device is an implantable device.

Described in certain example embodiments herein are methods of treating a nerve or neurodegenerative disease, disorder, and/or condition in a subject in need thereof, comprising implanting the device of the present disclosure comprising OSN derived EVs into the subject in need thereof.

A method of directing, increasing/enhancing axonal growth and/or the rate of axonal growth during neuron development and/or regeneration, the method comprising implanting the device of the present disclosure comprising OSN derived EVs into the subject in need thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B—Effect of neuron length per cell for dorsal root ganglions (DRGs) cultured without (FIG. 1A) or with (FIG. 1B) EVs derived from OSNs.

FIG. 2A-2G—Culturing primary olfactory neurons (FIG. 2A), purification of olfactory neuronal EVs (FIG. 2B) and their effect on OSNs from normal (FIG. 2C-2F) or OSNs from pcdh 19−/− mice (FIG. 2G).

FIG. 3—General structure of olfactory epithelium.

FIG. 4A-4B—Schematic of correct and incorrect glomeruli regeneration in the olfactory bulb after ablation by methimazole.

FIG. 5A-5B—Microscopic images demonstrating the olfactory epithelium after methimazole treatment at day 0 in saline control (FIG. 5A) and methimazole ablated (FIG. 5B) olfactory epithelium.

FIG. 6—OSN derived EVs promote olfactory epithelium regrowth.

FIG. 7—Microscopic (H &E stained) images of olfactory epithelium after methimazole ablation demonstrating sloughing followed by initial disorganized initial regeneration with no treatment with EVs.

FIG. 8A-8B—Microscopic (H &E stained) images of olfactory epithelium after control treated (saline) and OSN-derived EV treated olfactory epithelium.

FIG. 9—Fluorescent microscopic images of glomeruli at day 21 post ablation in saline treated, MI only (no control or EV treatment), or ablated+EV treated mice. EVs promoted better regrowth and neuron targeting to the olfactory bulb of olfactory neurons in vivo.

FIG. 10—General Nano LC MS/MS results.

FIG. 11A-11C—OSN derived EVs applied to cultured dorsal root ganglion (DRG) neurons increased neurite length.

FIG. 12A-12B—OSN derived EVs applied to transected sciatic nerves promoted improved axon organization during regeneration and may promote DRG regeneration.

FIG. 13A-13B—EV stock purification. EV stock collected from olfactory sensory neurons was successfully purified as shown by (+) flotillin (FIG. 13A) and (−) IkB detection (FIG. 13B).

FIG. 14—OSN layer condition comparative. The OSN layer for each condition's epithelium was measured at five randomized locations. Day 0 saline and MZ layer were significantly different (p<0.05), showing that MZ worked. Additionally, all EV conditions were significantly different from the day 14 saline condition (p<0.05).

FIG. 15A-15B—Day 0 imaging of olfactory epithelium. Both GFP imaging and hematoxylin and eosin (H&E) staining of the epithelium confirmed that methimazole (MZ) destroyed the olfactory sensory neuron (OSN) layer at day 0.

FIG. 16A-16D—H&E staining of the epithelium showed different cell patterns in the OSN layer within and across conditions. The MZ OSN layer appeared more unhealthy and loosely packed than the EV conditions. FIG. 16A. Methimazole only condition. FIG. 16B. 2 g EV condition. FIG. 16C. 4 μg EV condition. FIG. 16D. 8 μg EV condition.

FIG. 17A-17E—Fluorescent microscopic imaging of OSN axons under different conditions at day 14. FIG. 17A. Saline condition. FIG. 17B. Methimazole only condition. FIG. 17C. 2 μg EV condition. FIG. 17D. 4 μg EV condition. FIG. 17E. 8 μg EV condition. At day 14, all test condition treatments caused OSN axons to appear cloudy and not well defined as compared to saline treated OSN axons. At day 28, EV treated OSN axons had recovered more than untreated regenerating axons. 8 μg EV treated regenerating axons most closely resembled saline OSN axons.

FIG. 18A-18C—Experimental Apparatus: dissection and isolation of olfactory epithelium. FIG. 18A. Dorsal view of a neonatal mouse head. The line depicts the intended cut through the midsagittal plane. FIG. 18B. Schematic representation of mouse olfactory systems. FIG. 18C Sagittal view of an opened nasal cavity.

FIG. 19A-19C—Data analysis Apparatus: Immunostained sample images of neurons and ImageJ software for analysis of neurons. Immunostained images were analyzed (FIG. 19A) via ImageJ software, which traces neurons based on interpretation of where cell bodies and neurites are located. FIG. 19B shows the input and FIG. 19C shows the output, which ultimately converts into an excel sheet for statistical analysis.

FIG. 20A-20B—Western blot for verification of EV purification. FIG. 20A shows exposure of lysate and two EV samples to flotillin-2 primary antibody. FIG. 20B shows exposure of the lysate and one EV sample to IkB alpha primary antibody.

FIG. 21—Nanosight NS300 Nanoparticle Analysis for Secondary Verification of EV Purification. There are characteristic peaks (36 nm, 74 nm, and 128 nm) as well as smaller ones (183 nm, 372 nm, and 448 nm) that are all within expected ranges in a heterogenous population of EVs (both exosomes and microvesicles).

FIG. 22—Immunostained images of single concentration (8 ug) comparing EVD1 and EVD2 with Ctrl. Neurites are traced in red and cell bodies are stained with DAPI. Differences (arrows pointing out neurite extensions) can be observed between the two EV types with control with apparent greater growth of neuritelengths.

FIG. 23A-23B—Graphical Representation of Growth Distribution as a Measure of Total Length of Neurite Per Cell. At a selected concentration of 8 ug, both EVD1 and EVD2 showed significantly greater average total length of neurite compared to control (p<<<0.001); no significant differences were observed between the two EV populations (p=0.57) (FIG. 23A). To better visualize the distribution of data, a violin plot (FIG. 23B) (log of average total length of neurite) was made and a bimodal distribution was observed, albeit more prominent in the EV populations than the control. The line represents the mean.

FIG. 24—Comparison of Rab27DKO (Exosome-Deficient EVs) with normal EVs. All EV samples (normal and Rab27DKO) were significantly different than control for average total length of neurite (p<<<0.001). EVD1 and EVD2 were also significantly different than RabD1 and RabD1 (p<<0.01). There are no significant differences between EVD1 and EVD2 (p=0.56); similarly, no significant differences were noted between RabD1 and RabD2 (p=0.26).

FIG. 25—Effects of Electroporated Pcdh on Growth in OSNs. ‘P’ was used as a shorthanded abbreviation for Pcdh and P1_19 is the combined Pcdh1 and Pcdh19. There were significant differences in average neurite length compared to control for electroporated Pcdh19 and electroporated Pcdh1+19. No statistically significant differences, however, were observed for Pcdh1 compared to control (p=0.32).

FIG. 26A-26B—EV treated DRG cultures grew more, and longer neurites as compared to control treated DRG cultures.

FIG. 27A-27B—OSN derived EVs applied to transected sciatic nerves promoted improved axon organization during regeneration in vivo and may promote DRG regeneration.

FIG. 28—Schematic of technology for stamping EVs into patterns on coverslips or other suitable surfaces. After patterning, samples are dried on the surface.

FIG. 29—Microscopic image showing EVs and polyolefin pattern on a coverslip.

FIG. 30A-30B—Microscopic images demonstrating that OSN derived EVs patterned on a coverslip promote migration and extension of neurons along the pattern track.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g., 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g., plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one as used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “antibody” refers to a protein or glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen. “Antibody” includes single valent, bivalent and multivalent antibodies.

As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals.

As used herein, “aptamer” can refer to single-stranded DNA or RNA molecules that can bind to pre-selected targets including proteins with high affinity and specificity. Their specificity and characteristics are not directly determined by their primary sequence, but instead by their tertiary structure.

As used herein, “control” can refer to an alternative subject or sample used in an experiment for comparison purpose and included to minimize or distinguish the effect of variables other than an independent variable.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, the terms “disease” or “disorder” are used interchangeably throughout this specification, and refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition, or affliction.

As used herein, “dose,” “unit dose,” or “dosage” can refer to physically discrete units suitable for use in a subject, each unit containing a predetermined quantity of the delta protocadherin composition described herein and/or a pharmaceutical formulation thereof calculated to produce the desired response or responses in association with its administration.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein. As used herein, the term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

As used herein, “gene” can refer to a hereditary unit corresponding to a sequence of DNA that occupies a specific location on a chromosome and that contains the genetic instruction for a characteristic(s) or trait(s) in an organism. The term gene also refers to translated and/or untranslated regions of a genome. “Gene” can refer to the specific sequence of DNA that is transcribed into an RNA transcript that can be translated into a polypeptide or be a catalytic or other type of RNA molecule, including but not limited to, tRNA, siRNA, piRNA, miRNA, long-non-coding RNA and shRNA.

As used herein, “identity,” refers to a relationship between two or more nucleotide or polypeptide sequences, as determined by comparing the sequences. In the art, “identity” also refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides or polynucleotides of the present disclosure, unless stated otherwise.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

The term “modification causing said increased (or decreased) expression” refers to a modification in a gene which affects the expression level of that or another gene such that expression of that or another gene is increased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group of a mutation, a deletion and a frameshift. In particular embodiments, the modification is a mutation which results in increased or reduced expression of the functional gene product.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, “modulate” or “modify” and variations of such terms broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation-modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in aspects modulation may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In aspects, modulation may encompass a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In aspects, modulation may be specific or selective, hence, one or more desired phenotypic aspects of a cell or cell population may be modulated without substantially altering other (unintended, undesired) phenotypic aspect(s).

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used interchangeably herein, “operatively linked” and “operably linked” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g., promoters, enhancers, and termination elements), and/or selectable markers in an expression vector. “Operatively linked” can also refer to an indirect attachment (i.e., not a direct fusion) of two or more polynucleotide sequences or polypeptides to each other via a linking molecule (also referred to herein as a linker).

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be a three-dimensional network (e.g. the repeat units are linked together left and right, front and back, up and down), a two-dimensional network (e.g. the repeat units are linked together left, right, up, and down in a sheet form), or a one-dimensional network (e.g. the repeat units are linked left and right to form a chain). “Polymers” can be composed, natural monomers or synthetic monomers and combinations thereof. The polymers can be biologic (e.g., the monomers are biologically important (e.g., an amino acid), natural, or synthetic.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “purified” or “purify” can be used in reference to a nucleic acid sequence, peptide, or polypeptide that has increased purity relative to the natural environment. A purified compound, compounds, molecules, or other substance can have enhanced, improved, and/or substantially different properties and/or effects as compared to the compound(s) and/or molecules in its natural state.

As used herein, the term “radiation sensitizer” refers to agents that can selectively enhance the cell killing from irradiation in a desired cell population, such as tumor cells, while exhibiting no single agent toxicity on tumor or normal cells.

As used interchangeably herein “decreased expression”, “reduced expression”, or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control. The term “modification causing said reduced expression” refers to a modification in a gene which affects the expression level of that or another gene such that the expression level of that or another gene is reduced or decreased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

As used herein, “separated” can refer to the state of being physically divided from the original source or population such that the separated compound, agent, particle, or molecule can no longer be considered part of the original source or population.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸M or less, 10⁻⁹ M or less, 10⁻¹ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, object surface, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. For example, in some embodiments, the methods described herein include determining the disassociation constant for the targeting moiety and target. In some embodiments, the targeting moiety has a specificity the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, the targeting moiety has increased binding with, association with, interaction with, activity on as compared to non-targets, such as a 1 to 500 or more fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

As used herein, “therapeutic” refers to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a disease, disorder, condition and/or symptom thereof further described elsewhere herein. The effect can be therapeutic in terms of a partial or complete cure of a disease, condition, symptom or adverse effect attributed to the disease, disorder, or condition. The term “treatment” as used herein covers any treatment of a disease, disorder, condition and/or symptom thereof further described elsewhere herein, in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

As used herein in relation to polynucleotides and polypeptides, and more particularly to delta protocadherin genes and gene products, “diseased” refers to a mutant or modified variant of a wild-type or non-diseased variant that causes in whole or in part a disease, disorder, condition and/or a symptom thereof. In contrast, a non-diseased variant is a wild-type or variant thereof that does not cause, in whole or in part, a disease, disorder, condition and/or a symptom thereof.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

All publications, published patent documents, and patent applications cited herein are hereby incorporated by reference to the same extent as though each individual publication, published patent document, or patent application was specifically and individually indicated as being incorporated by reference.

### Overview

Current approaches to repair neuronal damage include microsurgery, glues to promote regrowth, and the development of conduits to bridge the proximal and distal stump (Xue, W., Shi, W., Kong, Y., Kuss, M., and Duan, B. Anisotropic scaffolds for peripheral nerve and spinal cord regeneration. Bioact. Mater., 2021, 6(11): 4141-60 and https://www.intechopen.com/chapters/55127). Another approach is to use cell-based therapies. In particular, the use of Schwann cells (a form of glia) is the most commonly studied such therapy (Hood, B., Levene, H. B., Levi, A. D. Transplantation of autologous Schwann cells for the repair of segmental peripheral nerve defects, Neurosurg. Focus, 2009 26: 1-9). Schwann cells enwrap axons, providing neurotrophic factors to support axon regrowth, myelin to protect and insulate axons, and modulate the immune response. However, they proliferate slowly and are difficult to obtain. Alternatives have included stem cells isolated from various sources, which are then differentiated into Schwann-cell like cells.

More recently, efforts aimed at identifying the factors produced by Schwann cells to promote regrowth suggest that extracellular vesicles (EVs) are important for this process. EVs are secreted by all cell-types, and contain RNAs, DNA, and protein. They have recently been identified as an important form of cell-cell communication. Uptake of EVs by cells can lead to significant changes in cellular homeostasis.

Schwann cell derived EVs were shown to promote the regrowth of peripheral neurons of neurons grown in culture and in rodents with sciatic nerve injury (Lopez-Verilli, M. A., Picou, F., Court, F. A. Schwann cell-derived exosomes enhance axonal regeneration in the peripheral nervous system Glia, 2013 61(11):1795-806). Other groups have also shown that EVs derived from olfactory ensheathing cells (another cell-type similar to Schwann cells and are also glial-like) also can promote regrowth of neurons in culture and in surgical models (Xia, B, Gao, J. Li, S. et al. Extracellular Vesicles Derived From Olfactory Ensheathing Cells Promote Peripheral Nerve Regeneration in Rats. Front. Cell. Neurosci., 2019, 13, Article 548). These results show glial culture-derived EVs are an important mechanism for promoting axonal regrowth both in culture and in vivo. Importantly, these studies using olfactory glial cells suggested that it was only the glial cells and/or EVs therefrom were the crucial cell type for proper olfactory neuron regeneration.

As previously discussed, the role of Schwann cells in promoting axonal regrowth has been extensively studied, and EVs derived from Schwann cells promote regrowth. In contrast, the role of neuron culture-derived EVs in axon growth has only been studied in a single paper (8). In this study, CNS (cortical) neuron culture-derived EVs inhibited growth of neurons grown in culture, and did not enhance regrowth (Gong, J., Korner, R., Gaitanos, L., Klein, R. Exosomes mediate cell contact-independent ephrin-Eph signaling during axon guidance. Journal of Cell Biol., 2016, 214(1): 35-44).

With that said, embodiments disclosed herein can olfactory sensory neuron (OSN) derived extracellular vehicles (EVs), formulations thereof, and uses thereof. In some embodiments, the OSN derived extracellular vesicles are effective to increase the growth of neurons during regeneration. In some embodiments, the OSN derived EVs and/or formulations thereof are used to promote nerve regeneration, particularly peripheral nerve regeneration, and to treat nerve injury. In some embodiments, a composition can be enriched for OSN derived EVs that express (natively or exogenously) cell adhesion molecules, particularly protocadherins, and more particularly delta protocadherins. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### OSN Extracellular Vesicles

Described in several example embodiments herein are compositions that include extracellular vesicles derived from and/or isolated from an olfactory sensory neuron and/or cell culture thereof. In some embodiments, the compositions include a population of olfactory sensory neuron (OSN) derived extracellular vesicles (EVs) enriched for EVs comprising and optionally expressing one or more cell adhesion molecules, wherein the one or more cell adhesion molecules are optionally one or more cadherins, one or more neural cell adhesion molecules (NCAMs), one or more epithelial cell adhesion molecule (EpCAM), one or more integrins, one or more plexins, or any combination thereof.

In certain example embodiments, the one or more cell adhesion molecules is/are, a delta protocadherin, optionally Pcdh1, Pcdh7, Pcdh8, Pcdh9, Pcdh10, Pcdh11, Pcdh17, Pcdh18, Pcdh19, Pcdh20 or any combination thereof.

In certain example embodiments, the OSN derived EVs are microvesicles, exosomes, or a heterogenous population of both.

In certain example embodiments, the OSN derived EVs comprise an exogenous cell adhesion molecule gene and/or gene product, wherein the exogenous cell adhesion molecule gene or gene product is optionally one or more cadherins, one or more neural cell adhesion molecules (NCAMs), one or more epithelial cell adhesion molecule (EpCAM), one or more integrins, one or more plexins, or any combination thereof. In some embodiments, such EVs are produced from genetically modified cells engineered to express the one or more exogenous cell adhesion molecules.

In certain example embodiments, the OSN derived EVs comprise an exogenous Pcdh1, Pcdh7, Pcdh8, Pcdh9, Pcdh10, Pcdh11, Pcdh17, Pcdh18, Pcdh19, or Pcdh20 gene or gene product, or any combination thereof.

In certain example embodiments, the composition is frozen, dehydrated, lyophilized, or otherwise modified for storage.

The compositions described herein can have one or more beneficial and/or therapeutic effects. In certain example embodiments, the composition is effective to stimulate axonal growth and/or increase the rate of axonal growth in a peripheral neuron, a central nervous system neuron, or both. In certain example embodiments, the composition is effective to increase or enhance correct axonal connectivity during neuron regeneration.

The EVs can be isolated as described in the Working Examples herein. In some embodiments, a cell or organism is modified to express one or more exogenous cell adhesion molecules as previously discussed using techniques generally known in the art. Such cells can be isolated and cultured as non-engineered cells and the EVs can be collected in the same fashion.

### Cargos

In certain example embodiments, the composition further comprises a cargo. Cargos can include any molecule that is capable of being contained in, integrated with, coupled to, or otherwise associated with the OSN EVs described herein. Suitable cargos include, without limitation, polynucleotides, polypeptides, nutrients (e.g., lipid, amino acid, carbohydrate, peptide, protein, sugar, vitamin, mineral, and/or the like), genetic modifying systems or component thereof, antibodies or fragments thereof, aptamers, affibodies, small molecule chemical agents (e.g., a therapeutic and/or prevention), immunomodulators, hormones, antipyretics, anxiolytics, antipsychotics, analgesics, antispasmodics, anti-epileptic agents, anti-inflammatory agents, anti-histamines, anti-infectives, radiation sensitizers, chemotherapeutics, a growth factors, neurotransmitter agonists, a neurotransmitter antagonists, or any combination thereof. Representative cargo molecules that may be delivered using the compositions disclosed herein include, but are not limited to, nucleic acids, polynucleotides, proteins, polypeptides, polynucleotide/polypeptide complexes, small molecules, sugars, or a combination thereof. Cargos that can be delivered in accordance with the systems and methods described herein include, but are not necessarily limited to, biologically active agents, including, but not limited to, therapeutic agents, imaging agents, and monitoring agents. A cargo may be an exogenous material or an endogenous material. In some embodiments, the cargo can be a “gene of interest”.

### Polynucleotides

In some embodiments, the cargo is a cargo polynucleotide. As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically, or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA, including but not limited to, tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), or coding mRNA (messenger RNA).

In some embodiments, the cargo polynucleotide is DNA. In some embodiments, the cargo polynucleotide is RNA. In some embodiments, the cargo polynucleotide is a polynucleotide (a DNA or an RNA) that encodes an RNA and/or a polypeptide. As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

### Genes of Interest

In some embodiments, the systems described herein comprise a polynucleotide encoding a gene of interest. As used herein, the term “gene of interest” refers to the gene selected for a particular purpose and being desired of delivery by a system or vesicle of the present invention. A gene of interest inserted into one or more regions a vector, such as an expression vector (including one or more of the engineered delivery vesicle generation system vectors) such that when expressed in a target cell or recipient cell it can be expressed and produce a desired gene product and/or be packaged as cargo in an engineered delivery vesicle of the present invention. It will be appreciated that other cargos specifically identified can also be genes of interest. For example, a polynucleotide encoding a Cas effector can be a gene of interest in this context where it is desired to deliver a Cas effector to a cell, for example.

In one embodiment, the gene of interest encodes a gene that provides a therapeutic function for the treatment of a disease. In some embodiments, the gene of interest can also be a vaccinating gene, that is to say a gene encoding an antigenic peptide that is capable of generating an immune response in humans or animals. This may include, but is not necessarily limited to, peptide antigens specific for viral and bacterial infections, or may be tumor-specific. In some embodiments, a gene of interest is a gene which confers a desired phenotype. As the embodiments described herein focus on improved methods for packaging and delivery of a gene of interest, the particular gene of interest is not limiting, and the technology can generally be used to deliver any gene of interest generally recognized by one of ordinary skill in the art as deliverable using a lentiviral system. One skilled in the art can design a construct containing any gene that they are interested in. Designing a construct containing a known gene of interest can be performed without undue experimentation. One of ordinary skill in the art routinely selects genes of interest. For example, the GenBank public database has existed since 1982 and is routinely used by persons of ordinary skill in the art relevant to the presently claimed method. As of June 2019, GenBank contains 2013,383,758 loci, 329,835,282,370 bases, from 213,383,758 reported sequences. The nucleotide sequences are from more than 300,000 organisms with supporting bibliographic and biological annotation. GenBank is only example, as there are many other known repositories of sequence information.

In some embodiments, the gene of interest may be, for example, a synthetic RNA/DNA sequence, a codon optimized RNA/DNA sequence, a recombinant RNA/DNA sequence (i.e., prepared by use of recombinant DNA techniques), a cDNA sequence or a partial genomic DNA sequence, including combinations thereof. Preferably, this is in the sense orientation. Preferably, the sequence is, comprises, or is transcribed from cDNA. The gene(s) of interest may also be referred to herein as “heterologous sequence(s)” “heterologous gene(s)” or “transgene(s)”.

In some embodiments, the gene of interest may confer some therapeutic benefit. The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

Preferably, the therapeutic agent may be administered in a therapeutically effective amount of the active components. The term “therapeutically effective amount” refers to an amount which can elicit a biological or medicinal response in a tissue, system, animal, or human that is being sought by a researcher, veterinarian, medical doctor or other clinician, and in particular can prevent or alleviate one or more of the local or systemic symptoms or features of a disease or condition being treated. In some embodiments, the disease or condition is a disease or condition of or affecting nerves, neurons, the PNS or CNS, or cell thereof. Exemplary diseases and disorders are described in greater detail elsewhere herein. In some embodiments, the gene of interest may lead to modified expression in the target cell.

In some embodiments, the gene of interest is a cell adhesion molecule or functional domain thereof. In some embodiments, the gene of interest is a delta protocadherin molecule or functional domain thereof. In some embodiments, the gene of interest is one or more cadhereins, one or more neural cell adhesion molecules (NCAMs), one or more epithelial cell adhesion molecule (EpCAM), one or more integrins, one or more plexins, or any combination thereof. In certain example embodiments, the gene of interest is a delta protocadherin, optionally Pcdh1, Pcdh7, Pcdh8, Pcdh9, Pcdh10, Pcdh11, Pcdh17, Pcdh18, Pcdh19, or Pcdh20 or any combination thereof.

### Genetic Modifying Agents

In certain embodiments, the one or more modulating agents may be a genetic modifying agent. The genetic modifying agent may comprise a programmable nuclease system (e.g., an RNA-guided system (e.g., a CRISPR (also referred to herein as a CRISPR-Cas system), a zinc finger nuclease system, a TALEN, a meganuclease), an RNAi system (e.g., antisense RNA, siRNA, and CRISPRi), RNA editors, or a combination thereof. In some embodiments, a delta protocadherin gene or gene product can be modified with the genetic modifying agents. Modifications are discussed elsewhere herein. In some embodiments, the gene that is modified is a one or more cadhereins, one or more neural cell adhesion molecules (NCAMs), one or more epithelial cell adhesion molecule (EpCAM), one or more integrins, one or more plexins, or any combination thereof. In certain example embodiments, the gene or gene product modified is a delta protocadherin, optionally Pcdh1, Pcdh7, Pcdh8, Pcdh9, Pcdh10, Pcdh11, Pcdh17, Pcdh18, Pcdh19, or Pcdh20 or any combination thereof. Modifications include, without limitation, deletions, substitutions, insertions, frameshift mutations, and/or the like.

### CRISPR-Cas Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

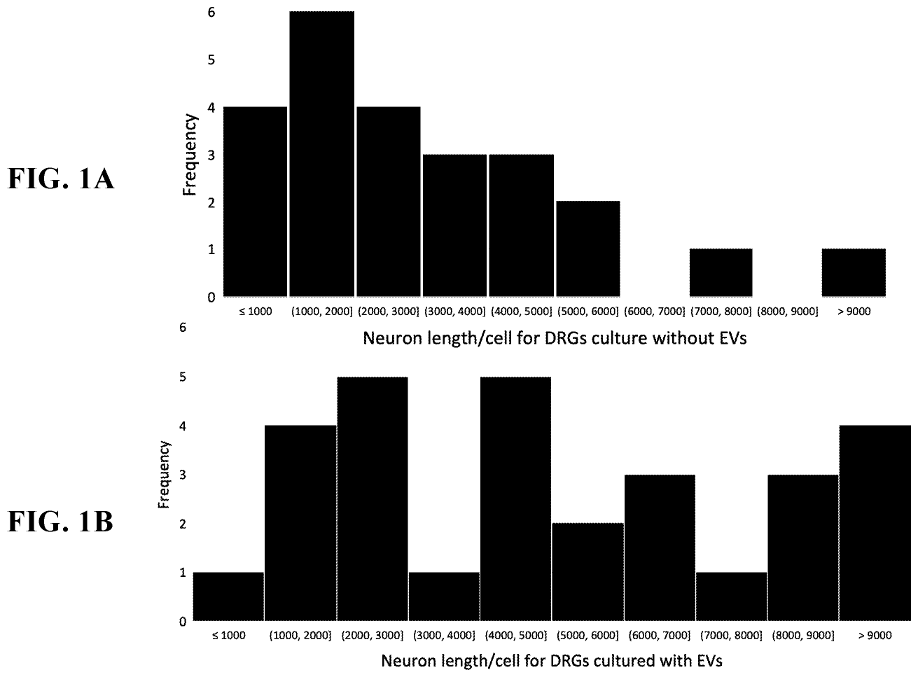

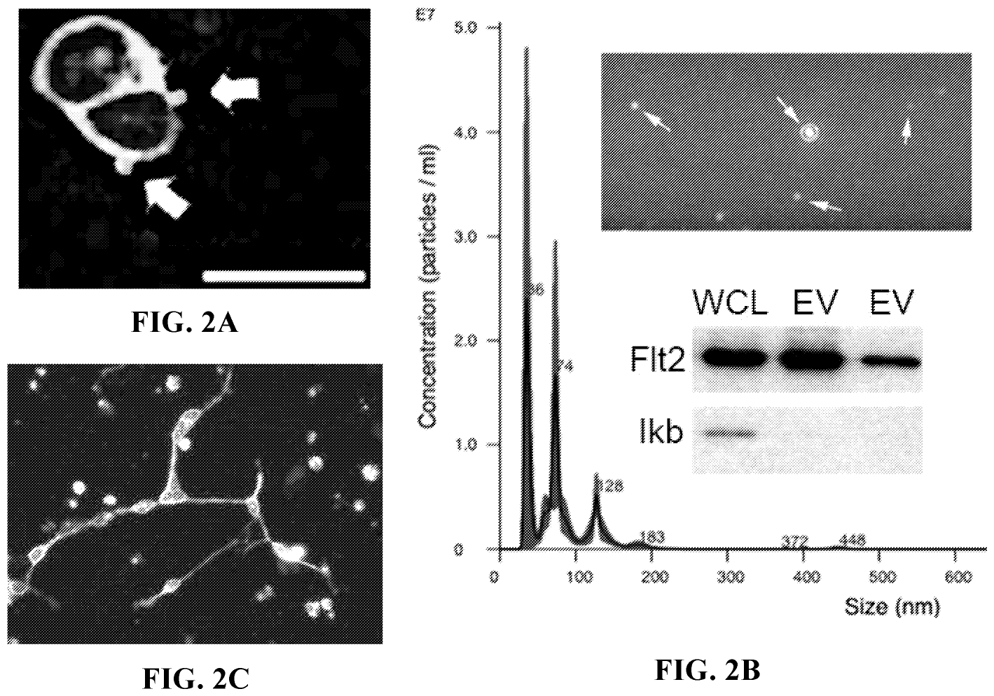

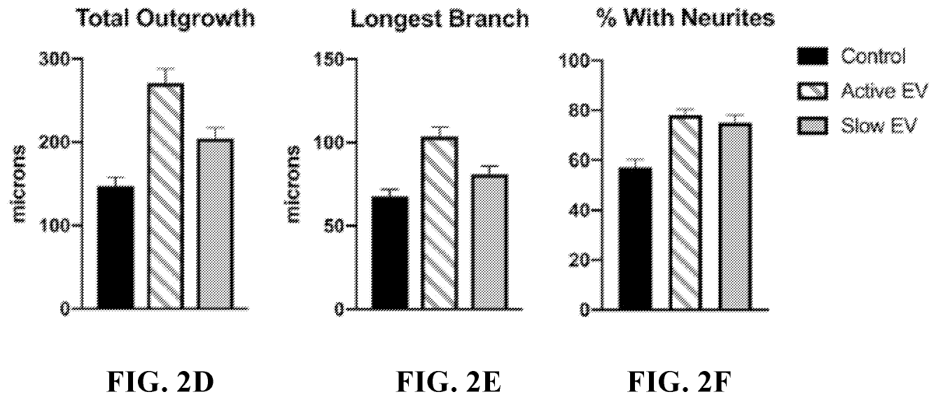

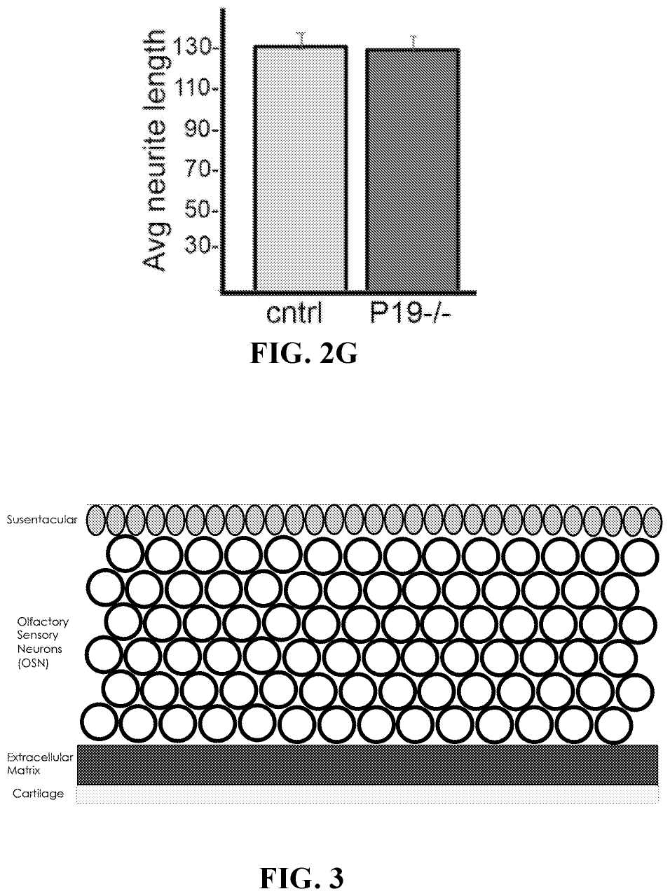

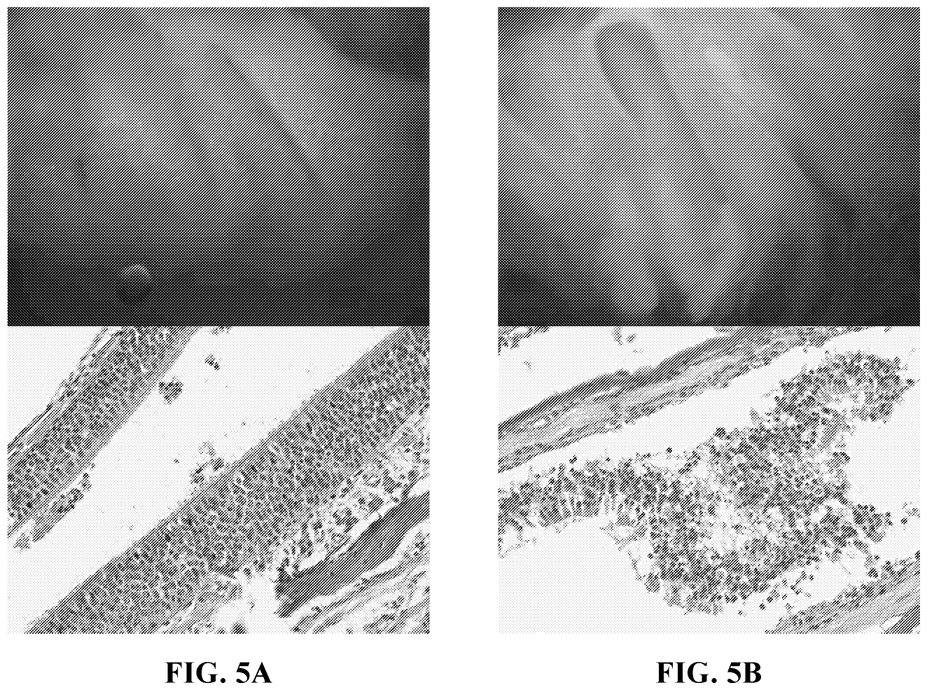

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020)., incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 1 (from Gleditzsch et al. 2019) below shows several Cas polypeptides and the PAM sequence they recognize.

TABLE 1
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples ofNLSs include an NLS sequence derived from: the NLS of the SV40 virus large T-antigen, having the amino acid sequence PKKKRKV (SEQ ID NO: 1) or PKKKRKVEAS (SEQ ID NO: 2); the NLS from nucleoplasmin (e.g., the nucleoplasmin bipartite NLS with the sequence KRPAATKKAGQAKKKK (SEQ ID NO: 3)); the c-myc NLS having the amino acid sequence PAAKRVKLD (SEQ ID NO: 4) or RQRRNELKRSP (SEQ ID NO: 5); the hRNPA1 M9 NLS having the sequence NQSSNFGPMKGGNFGGRSSGPYGGGGQYFAKPRNQGGY (SEQ ID NO: 6); the sequence RMRIZFKNKGKDTAELRRRRVEVSVELRKAKKDEQILKRRNV (SEQ ID NO: 7) of the IBB domain from importin-alpha; the sequences VSRKRPRP (SEQ ID NO: 8) and PPKKARED (SEQ ID NO: 9) of the myoma T protein; the sequence PQPKKKPL (SEQ ID NO: 10) of human p53; the sequence SALIKKKKKMAP (SEQ ID NO: 11) of mouse c-abl IV; the sequences DRLRR (SEQ ID NO: 12) and PKQKKRK (SEQ ID NO: 13) of the influenza virus NS1; the sequence RKLKKKIKKL (SEQ ID NO: 14) of the Hepatitis virus delta antigen; the sequence REKKKFLKRR (SEQ ID NO: 15) of the mouse Mx1 protein; the sequence KRKGDEVDGVDEVAKKKSKK (SEQ ID NO: 16) of the human poly(ADP-ribose) polymerase; and the sequence RKCLQAGMNLEARKTKK (SEQ ID NO: 17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Donor Templates

In some embodiments, the CRISPR-Cas system includes a donor template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 1 1, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g., VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing Systems

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system. See e.g., Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

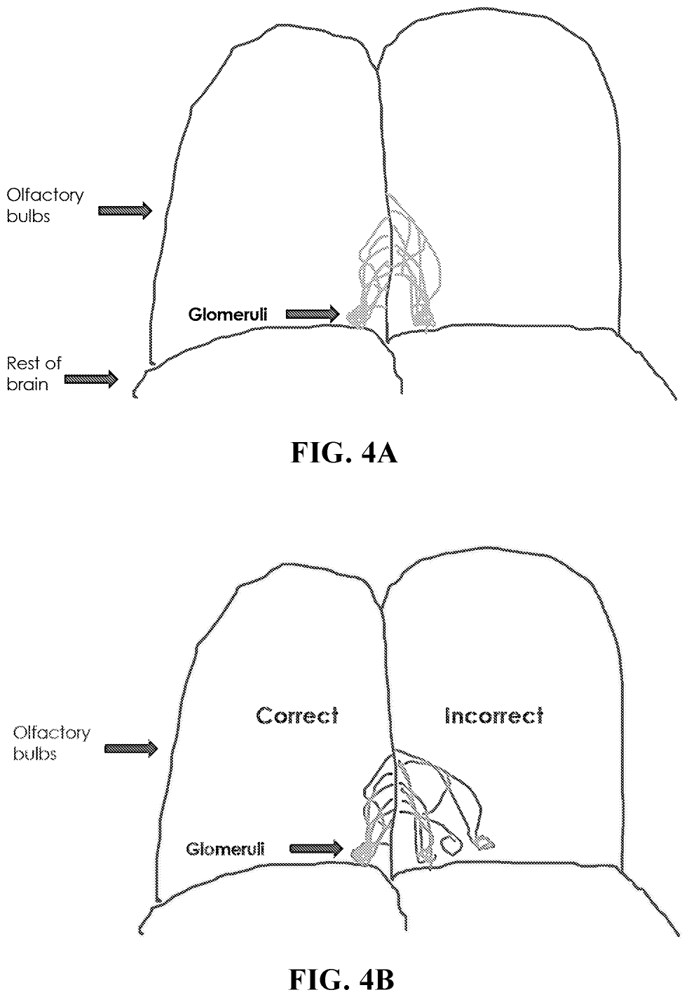

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 18)
MDPIRSRTPSPARELLSGPQPDGVQPTADRGVSPPAGGPLDGLPARRTMS
RTRLPSPPAPSPAFSADSFSDLLRQFDPSLFNTSLFDSLPPFGAHHTEAA
TGEWDEVQSGLRAADAPPPTMRVAVTAARPPRAKPAPRRRAAQPSDASPA
AQVDLRTLGYSQQQQEKIKPKVRSTVAQHHEALVGHGFTHAHIVALSQHP
AALGTVAVKYQDMIAALPEATHEAIVGVGKQWSGARALEALLTVAGELRG
PPLQLDTGQLLKIAKRGGVTAVEAVHAWRNALTGAPLN

An exemplary amino acid sequence of a C-terminal capping region is:

(SEQ ID NO: 19)
RPALESIVAQLSRPDPALAALTNDHLVALACLGGRPALDAVKKGLPHAPA
LIKRTNRRIPERTSHRVADHAQVVRVLGFFQCHSHPAQAFDDAMTQFGMS
RHGLLQLFRRVGVTELEARSGTLPPASQRWDRILQASGMKRAKPSPTSTQ
TPDQASLHAFADSLERDLDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Zinc Finger Nucleases

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is an RNA interference (RNAi) system (e.g., shRNA, antisense RNA, CRISPRi and/or the like). Such systems act to inhibit or in some configurations activate transcription and/or translation of a polynucleotide such that expression of the polynucleotide is reduced and/or functionally eliminated. Various mechanisms of action are employed by different systems to accomplish transcription and/or translation inhibition. As used herein, “gene silencing” or “gene silenced” in reference to an activity of an RNAi molecule or system where transcription and/or translation is inhibited or repressed such that expression of the gene is reduced, optionally to levels where no gene transcription or translation can be detected.

As used herein, the term “RNAi” refers to any type of interfering RNA or system that interferes with RNA transcription or translation, including but not limited to, siRNAi, shRNAi, endogenous microRNA and artificial microRNA, antisense RNA, CRISPRi, and/or the like. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

Any suitable RNAi molecule or system cans be used to modify the expression of a delta protocadherin gene. Such molecules and systems are generally known in the art and include without limitation, siRNA, shRNA, microRNA, piRNA, CRISPRi, antisense RNA, long non-coding RNA, and/or the like. See e.g., Setten et al., 2019, Nat Rev Drug Discov. 2019 June; 18(6):421-446. doi: 10.1038/s41573-019-0017-4; K. Lundstrom. Viruses. 2020 Aug. 23; 12(9):924. doi: 10.3390/v12090924; Saw et al., Sci China Life Sci. 2020 April; 63(4):485-500. doi: 10.1007/s11427-018-9438-y; Bajan et al. Cells. 2020 Jan. 7; 9(1):137. doi: 10.3390/cells9010137; Weng et al., Biotechnol Adv. 2019 September-October; 37(5):801-825. doi: 10.1016/j.biotechadv.2019.04.012; Dong et al., Adv Drug Deliv Rev. 2019 April; 144:133-147. doi: 10.1016/j.addr.2019.05.004; Hu et al., Signal Transduct Target Ther. 2020 Jun. 19; 5(1):101. doi: 10.1038/s41392-020-0207-x; Sajid et al., Pharmaceuticals (Basel). 2020 Oct. 7; 13(10):294. doi: 10.3390/ph13100294; Hsu et al., Biotechnol Adv. 2019 December; 37(8):107447. doi: 10.1016/j.biotechadv.2019.107447; Peddle et al., Int J Mol Sci. 2020 Mar. 27; 21(7):2329. doi: 10.3390/ijms21072329; Adiego-Perez et al., FEMS Microbiol Lett. 2019 Apr. 1; 366(8):fnz086. doi: 10.1093/femsle/fnz086; Anton et al., Biol Methods Protoc. 2018 May 10; 3(1):bpy002. doi: 10.1093/biomethods/bpy002; Nishida and Kondo. Metab Eng. 2021 January; 63:141-147. doi: 10.1016/j.ymben.2020.12.002; Kondrateva et al., Gene. 2021 Feb. 15; 769:145225.doi: 10.1016/j.gene.2020.145225, which are incorporated by reference herein and can be adapted for use with the present embodiments.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Therapeutic Polynucleotides

In some embodiments, the cargo molecule is a therapeutic polynucleotide. Therapeutic polynucleotides are those that provide a therapeutic effect when delivered to a recipient cell. The polynucleotide can be a toxic polynucleotide (a polynucleotide that when transcribed or translated results in the death of the cell) or polynucleotide that encodes a lytic peptide or protein. In embodiments, delivery vesicles having a toxic polynucleotide as a cargo molecule can act as an antimicrobial or antibiotic. This is discussed in greater detail elsewhere herein. In some embodiments, the cargo molecule can be exogenous to the producer cell and/or a first cell. In some embodiments, the cargo molecule can be endogenous to the producer cell and/or a first cell. In some embodiments, the cargo molecule can be exogenous to the recipient cell and/or a second cell. In some embodiments, the cargo molecule can be endogenous to the recipient cell and/or second cell.

As described herein the cargo polynucleotide can be any polynucleotide endogenous or exogenous to the eukaryotic cell. For example, the cargo polynucleotide can be a polynucleotide residing in the nucleus of the eukaryotic cell. The cargo polynucleotide can be a sequence coding a gene product (e.g., a protein) or a non-coding sequence (e.g., a regulatory polynucleotide).

In some embodiments, the cargo polynucleotide is a DNA or RNA (e.g., a mRNA) vaccine.

### Aptamers

In certain example embodiments, the polynucleotide may be an aptamer. In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues, and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10⁻¹⁵ kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH2), 2′-fluoro (2′-F), and/or 2′-O-methyl (2′-OMe) substituents. Modifications of aptamers may also include modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to, those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see e.g., SomaLogic, Inc., Boulder, Colorado). In certain embodiments, the present invention may utilize any aptamer containing any modification as described herein.

In certain other example embodiments, the polynucleotide may be a ribozyme or other enzymatically active polynucleotide.

### Biologically Active Agents

In some embodiments, the cargo is a biologically active agent. Biologically active agents include any molecule that induces, directly or indirectly, an effect in a cell. Biologically active agents may be a protein, a nucleic acid, a small molecule, a carbohydrate, and a lipid. When the cargo is or comprises a nucleic acid, the nucleic acid may be a separate entity from the DNA-based carrier. In these embodiments, the DNA-based carrier is not itself the cargo. In other embodiments, the DNA-based carrier may itself comprise a nucleic acid cargo. Therapeutic agents include, without limitation, chemotherapeutic agents, anti-oncogenic agents, anti-angiogenic agents, tumor suppressor agents, anti-microbial agents, enzyme replacement agents, gene expression modulating agents and expression constructs comprising a nucleic acid encoding a therapeutic protein or nucleic acid, and vaccines. Therapeutic agents may be peptides, proteins (including enzymes, antibodies and peptidic hormones), ligands of cytoskeleton, nucleic acid, small molecules, non-peptidic hormones and the like. To increase affinity for the nucleus, agents may be conjugated to a nuclear localization sequence. Nucleic acids that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, expression constructs, etc.

Imaging agents include contrast agents, such as ferrofluid-based MRI contrast agents and gadolinium agents for PET scans, fluorescein isothiocyanate and 6-TAMARA. Monitoring agents include reporter probes, biosensors, green fluorescent protein, and the like. Reporter probes include photo-emitting compounds, such as phosphors, radioactive moieties, and fluorescent moieties, such as rare earth chelates (e.g., europium chelates), Texas Red, rhodamine, fluorescein, FITC, fluo-3, 5 hexadecanoyl fluorescein, Cy2, fluor X, Cy3, Cy3.5, Cy5, Cy5.5, Cy7, dansyl, phycocrytherin, phycocyanin, spectrum orange, spectrum green, and/or derivatives of any one or more of the above. Biosensors are molecules that detect and transmit information regarding a physiological change or process, for instance, by detecting the presence or change in the presence of a chemical. The information obtained by the biosensor typically activates a signal that is detected with a transducer. The transducer typically converts the biological response into an electrical signal. Examples of biosensors include enzymes, antibodies, DNA, receptors, and regulator proteins used as recognition elements, which can be used either in whole cells or isolated and used independently (D'Souza, 2001, Biosensors and Bioelectronics 16:337-353).

### Hormones

In certain embodiments, the one or mor polynucleotides may comprise one or more hormones. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g. CSH1, CHS2), testosterone. and neuroendocrine hormones. In certain examples, the hormone is secreted from pancreas, e.g., insulin, glucagon, somatostatin, pancreatic polypeptide and ghrelin. In some examples, the hormone is insulin.

Hormones herein may also include growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, and glucocorticoids. In a particular embodiment, the hormone is insulin or incretins such as exenatide, GLP-1.

### Neurohormones

In embodiments, the secreted peptide is a neurohormone, a hormone produced and released by neuroendocrine cells. Example neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

### Small Molecules

In some embodiments, the cargo molecule is a small molecule. Techniques and methods of coupling peptides to small molecule agents are generally known in the art and can be applied here to couple a targeting moiety effective to target a CNS cell to a small molecule cargo. Small molecules include, without limitation, hormones, immunomodulators, antipyretics, anxiolytics, antipsychotics, analgesics, antispasmodics, anti-inflammatories, anti-histamines, anti-infectives, radiation sensitizers, chemotherapeutics.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosteron Cortisol). Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-a, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g., ibuprofen, naproxen, ketoprofen, and nimesulide), aspirin and related salicylates (e.g., choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotenergic antidepressants (e.g., selective serotonin reuptake inhibitors, tricyclic antidepresents, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, abendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telvancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g., lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicyclic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

### Targeting Moieties

In certain example embodiments, the OSN derived EVs include or are otherwise associated with one or more targeting moieties, wherein the targeting moiety is optionally a peptide, polypeptide, polynucleotide, sugar, a chemical molecule, a polymer, a lipid, a glycan, a peptidoglycan, or any combination or complex thereof (e.g., receptors, receptor ligands, antibodies and fragments thereof, aptamers, affibodies, antibody and/or aptamer epitopes, binding agents and their binding partners (e.g., biotin and streptavidin, enzymes and their substrates, a targeting nucleic acid, target nucleic acid and guided nuclease (e.g., miRNA, gRNA, RISC, Cas, etc.), guide nucleic acid for a guided nuclease system, and/or the like.

The targeting moieties can function to direct (or target) the EVs to one or more cell types so as to provide selective delivery of the EVs. In some embodiments, the targeting moiety is capable of targeting the EV to a nerve cell or neuron.

### Modified Cells and Organisms

Described herein are modified cells that have been modified to express one or more one or more cell adhesion molecules, wherein the one or more cell adhesion molecules are optionally one or more cadherins, one or more neural cell adhesion molecules (NCAMs), one or more epithelial cell adhesion molecule (EpCAM), one or more integrins, one or more plexins, or any combination thereof. The cells may be further modified to produce a cargo molecule which can then be incorporated into an EV produced by the modified cell. The cells that are modified can be any suitable mammalian cell. In some embodiments, the cells that are modified are olfactory cells. In some embodiments the cells that are modified are neurons. In some embodiments, the cells that are modified are neuroglial or glial cells (e.g., astrocytes, microglia, oligodendrocytes, radial glial cells, Schwann cells, ensheathing cells, and/or the like), astrocytes. In some embodiments, the olfactory cells that are modified are olfactory neurons or olfactory glial cells. The cells can be modified using any suitable genetic modification technique and or system, including but not limited to those described elsewhere herein. Others will be appreciated by those of ordinary skill in the art in view of the description herein.

The modified cells can be produced by a modified organism. Thus, also described herein are modified organisms, such as non-human mammalian species (including but not limited to non-human primates) that are modified to contain one or more cells, particularly one or more neurons, and more particularly one or more olfactory neuron cells, that express or over express one or more one or more cell adhesion molecules, wherein the one or more cell adhesion molecules are optionally one or more cadherins, one or more neural cell adhesion molecules (NCAMs), one or more epithelial cell adhesion molecule (EpCAM), one or more integrins, one or more plexins, or any combination thereof.

Methods of preparing EVs from the modified cells are the same as non-modified cells, which are described in greater detail elsewhere herein.

In some embodiments, the modified cells can be included in a pharmaceutical formulation described elsewhere herein and/or administered to a subject in need thereof where they can produce the therapeutic EVs described elsewhere herein.

### Formulations

### Pharmaceutical Formulations

Also described herein are pharmaceutical formulations that can contain an amount, effective amount, and/or least effective amount, and/or therapeutically effective amount of one or more compositions (which includes in this context all the compositions and cells, such as the modified cells) described elsewhere herein) (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient an OSN EV or population thereof as described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, the compositions described in greater detail elsewhere herein can be provided to a subject in need thereof as an ingredient, such as an active ingredient or agent, in a pharmaceutical formulation. Where appropriate, a compound or composition contained in the in the formulation can be formulated as a pharmaceutically acceptable salt thereof. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has, has had, and/or is suspected of having a nerve injury, nerve death, aberrant neuron connectivity, aberrant neuron activity, a neuropathy, or any combination thereof. In some embodiments, the subject in need thereof has, has had, and/or is suspected of having a neurodegenerative disease, disorder, and/or condition. In some embodiments, the subject in need thereof has, has had, and/or is suspected of having an epilepsy, a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, and/or is suspected of having a CNS neuron/nerve and/or a peripheral neuron/nerve injury, disease, disorder, and/or condition.

In some embodiments, the subject in need thereof has, has had, and/or is suspected of having an epilepsy, a seizure disease, disorder or condition, or a disease, disorder, or condition in which seizures are a symptom or result of the disease, disorder, or condition, including but not limited to non-epileptic seizures. In some embodiments, the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

In some embodiments, the subject in need thereof has, has had, or is suspected of having a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, or is suspected of having a CNS neuron/nerve and/or a peripheral neuron/nerve injury, disease, disorder, and/or condition. In some embodiments, the disease, disorder, and/or condition is a genetic disease, disorder, and/or condition. In some embodiments, the disease, disorder, and/or condition is not a genetic disease, disorder, and/or condition.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules (including, but not limited to, e.g. polynucleotides, amino acids, peptides, polypeptides, antibodies and fragments thereof, aptamers, ribozymes, hormones, and/or the like), affibodies, immunomodulators, antipyretics, anxiolytics, antipsychotics, analgesics, antispasmodics, anti-inflammatories, anti-histamines, anti-infectives, an anti-epileptic agent, neurotransmitter agonists, neurotransmitter antagonists, chemotherapeutics, a nutrient (e.g., lipid, amino acid, carbohydrate, peptide, protein, sugar, vitamin, mineral, and/or the like), a small molecule chemical agent (e.g., a therapeutic or prevention), genetic modifying system or component thereof (e.g., a CRISPR-Cas system, Zinc Finger Nuclease system, a Mega nuclease system, an ADAR, a base editing system, RNAi or other gene silencing system, and/or the like), and any combination thereof. In some embodiments, the secondary agent is formulated in an EV, such as a cargo. In some embodiments, the secondary agent is contained in the formulation but is not contained in the EV.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are increased neuron growth and/or regeneration, increased axon length, growth, and/or regeneration, and/or increased rate of neuron and/or axon growth and/or regeneration. In some embodiments, the one or more therapeutic effects are increased correct axon connectivity. This refers to the axon connecting to the appropriate target neuron or neurons during regeneration so as to be more similar to and/or like a pre-disease or pre-injured/damaged state. In some embodiments, the one or more therapeutic effects is or includes improved neuron and/or axon structure and organization during regeneration. This refers to the overall alignment, spacing, and/or positioning of the regenerating neurons so as to be more similar to and/or like a pre-disease or pre-injured/damaged state.

The effective amount, least effective amount, and/or therapeutically effective amount of the primary and optional secondary active agent described elsewhere herein contained in the pharmaceutical formulation, when present, can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, g, mg, or g or be any numerical value with any of these ranges.

In some embodiments, the effective amount, least effective amount, and/or therapeutically effective amount can be an effective concentration, least effective concentration, and/or therapeutically effective concentration of the primary and optional secondary active agent described elsewhere herein contained in the pharmaceutical formulation, when present, can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, M, mM, or M or be any numerical value with any of these ranges.

In other embodiments, the effective amount, least effective amount, and/or therapeutically effective amount of the primary and optional secondary active agent described elsewhere herein contained in the pharmaceutical formulation, when present, can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent when present in the pharmaceutical formulation can be present at any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where an EV or cell population is present in the pharmaceutical formulation (e.g., as a primary and/or or secondary active agent), the effective amount of EVs or cells can range from about 2 EVs or cells to 1×10¹ EVs or cells/mL, 1×10¹⁰⁰ EVs or cells/mL or more, such as about 1×10¹ EVs or cells/mL, 1×10² EVs or cells/mL, 1×10³/EVs or cells/mL, 1×10⁴ EVs or cells/mL, 1×10⁵ EVs or cells/mL, 1×10⁶ EVs or cells/mL, 1×10⁷ EVs or cells/mL, 1×10⁸ EVs or cells/mL, 1×10⁹ EVs or cells/mL, 1×10¹⁰ EVs or cells/mL, 1×10¹¹ EVs or cells/mL, 1×10¹ EVs or cells/mL, 1×10¹³ EVs or cells/mL, 1×10¹⁴ EVs or cells/mL, 1×10¹⁵ EVs or cells/mL, 1×10¹⁶ EVs or cells/mL, 1×10¹⁷ EVs or cells/mL, 1×10¹⁸ EVs or cells/mL, 1×10¹⁹ EVs or cells/mL, 1×10²⁰ EVs or cells/mL, 1×10³⁰ EVs or cells/mL, 1×10⁴⁰ EVs or cells/mL, 1×10⁵⁰ EVs or cells/mL, 1×10⁷⁰ EVs or cells/mL, 1×10⁶⁰ EVs or cells/mL, 1×10⁸⁰ EVs or cells/mL, 1×10⁹⁰ EVs or cells/mL, or/to about 1×10¹⁰⁰ EVs or cells/mL or more.

In some embodiments, the amount or effective amount, particularly where an infective particle is being delivered (e.g., a virus particle carrying an active agent), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, pL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, MD, 2000, and “Pharmaceutical dosage forms and drug delivery systems”, 6th Edition, Ansel et al., (Media, PA: Wlliams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome or EV. In these embodiments, primary active ingredient(s), and/or optional secondary active ingredient(s), and/or pharmaceutically acceptable salt thereof where appropriate are incorporated into a liposome or EV. In embodiments where the dosage form is a liposome or EV, the pharmaceutical formulation is thus a liposomal or EV formulation. The liposomal or EV formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulation. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

In some embodiments, the dosage form is adapted for targeted delivery to a peripherial nerve, such as using any of the approaches discussed in e.g., Langert and Brey (Front. Neurosci. 2018 https://doi.org/10.3389/fnins.2018.00887), which is incorporated by reference herein.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, a polynucleotide, a polypeptide, a nutrient (e.g., lipid, amino acid, carbohydrate, peptide, protein, sugar, vitamin, mineral, and/or the like), genetic modifying system or component thereof, antibody or fragment thereof, aptamer, ribozymes, affibody, small molecule chemical agent (e.g., a therapeutic and/or prevention), an immunomodulator, a hormone, an antipyretic, an anxiolytic, an antipsychotic, an analgesic, an antispasmodic, an anti-inflammatory agent, an anti-epileptic agent, an anti-histamine, an anti-infective, a growth factor, a radiation sensitizer, a chemotherapeutic, a neurotransmitter agonist, a neurotransmitter antagonist, or any combination thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Devices

In some embodiments, EVs, are be stamped and patterned on the surface of a device. The device can be implanted in a subject, such as where nerve regrowth is desired. In some embodiments, the EVs are dried on the surface of the implant in the desired pattern. In some embodiments, the EVs are patterned along with one or more polymers, such as a biocompatible polymer. In some embodiments, the polymer is a poly olefin. In some embodiments, the patterns on the device can be designed such that they form correct or desired nerve tracts. Without being bound by theory, when the device is implanted into a subject the EVs device can stimulate nerve (e.g., axon) growth along the pattern where the EVs are present. Without being bound by theory, nerve growth can be controlled or directed by how the EVs are patterned on the device. In some embodiments, the devices can be used in a method of treating subject and/or promoting nerve growth by implanting them in a subject in need thereof. In some embodiments, the devices are implanted at or in proximity to a damaged or otherwise injured or dysfunctional nerve.

In some embodiments, the device is made of one or more flexible materials. In some embodiments, the device is or includes one or more meshes, stents, tubes, planar surfaces, curved members. In some embodiments, the device is or includes one or more fibrous substrates. In some embodiments, the device comprises one or more biocompatible polymers.

In some embodiments, the device is suitable for in vivo use. In some embodiments, the device is configured for cell culture use.

### Kits

Any of the compounds, compositions, formulations, particles, cells, and/or devices described herein, or a combination thereof can be presented as a combination kit. As used herein, the terms “combination kit” or “kit of parts” refers to the compounds, compositions, formulations, particles, cells, and/or devices and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet, solution, suspension, powder, and/or the like described elsewhere herein) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof.

In some embodiments, the subject in need thereof has, has had, and/or is suspected of having a nerve injury, nerve death, aberrant neuron connectivity, aberrant neuron activity, a neuropathy, or any combination thereof. In some embodiments, the subject in need thereof has, has had, and/or is suspected of having a neurodegenerative disease, disorder, and/or condition. In some embodiments, the subject in need thereof has, has had, and/or is suspected of having an epilepsy, a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, and/or is suspected of having a CNS neuron/nerve and/or a peripheral neuron/nerve injury, disease, disorder, and/or condition.

In some embodiments, the subject in need thereof has, has had, and/or is suspected of having an epilepsy, a seizure disease, disorder or condition, or a disease, disorder, or condition in which seizures are a symptom or result of the disease, disorder, or condition, including but not limited to non-epileptic seizures. In some embodiments, the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

In some embodiments, the subject in need thereof has, has had, or is suspected of having a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, or is suspected of having a CNS neuron/nerve and/or a peripheral neuron/nerve injury, disease, disorder, and/or condition. In some embodiments, the disease, disorder, and/or condition is a genetic disease, disorder, and/or condition. In some embodiments, the disease, disorder, and/or condition is not a genetic disease, disorder, and/or condition.

### Methods of Using the OSN Extracellular Vesicles

The OSN EVs described in the several exemplary embodiments herein and formulations thereof can be used to treat a disease, disorder, condition, and/or injury. In some embodiments, a method of treating a disease, disorder, condition, and/or injury includes administering to a subject in need thereof a population and/or an amount of OSN EVs described in greater detail elsewhere herein to the subject in need thereof.

The OSN EVs described in the several exemplary embodiments herein and formulations thereof can be used to regenerate neurons and/or nerves in the CNS and/or periphery. In some embodiments, the OSN EVs described in the several exemplary embodiments herein and formulations thereof can be used to increase growth and/or regeneration of a neuron or nerve in the CNS and/or periphery. In some embodiments, a method of regenerating neurons and/or nerves includes administering to a subject in need thereof a population and/or an amount of OSN EVs described in greater detail elsewhere herein to the subject in need thereof.

In some embodiments, the OSN EVs described in the several exemplary embodiments herein and formulations thereof can be used to increase growth rate and/or regeneration rate of a neuron or nerve in the CNS and/or periphery. In some embodiments, the OSN EVs described in the several exemplary embodiments herein and formulations thereof can be used to increase growth rate and/or regeneration rate of an axon in the CNS and/or periphery. In some embodiments, a method of increasing the growth rate and/or regeneration rate of neurons and/or nerves includes administering to a subject in need thereof a population and/or an amount of OSN EVs described in greater detail elsewhere herein to the subject in need thereof.

In some embodiments, the OSN EVs or formulations thereof described in the several exemplary embodiments herein can be used to increase and/or promote axon and/or synapse formation, connectivity, or both. In some embodiments, the OSN EVs or formulations thereof described in the several exemplary embodiments herein can be used to increase and/or promote CNS or periphery axon and/or synapse formation, connectivity, or both. In some embodiments, a method of increase and/or promote axon and/or synapse formation, connectivity, or both includes administering to a subject in need thereof a population and/or an amount of OSN EVs described in greater detail elsewhere herein to the subject in need thereof.

The OSN EVs described in the several exemplary embodiments herein and formulations thereof can be used to increase correct axon connectivity during growth and/or regeneration. This refers to the axon connecting to the appropriate target neuron or neurons during regeneration so as to be more similar to and/or like a pre-disease or pre-injured/damaged state. In some embodiments, a method of increasing the correct connectivity of neurons and/or nerves, particularly the axon thereof, particularly during growth and/or regeneration, includes administering to a subject in need thereof a population and/or an amount of OSN EVs described in greater detail elsewhere herein to the subject in need thereof.

The OSN EVs described in the several exemplary embodiments herein and formulations thereof can be used to improve neuron and/or axon structure and organization during growth and/or regeneration. This refers to the overall alignment, spacing, and/or positioning of the regenerating neurons so as to be more similar to and/or like a pre-disease or pre-injured/damaged state. In some embodiments, a method of improving neuron/nerve and/or axon structure and organization, particularly during growth and/or regeneration, includes administering to a subject in need thereof a population and/or an amount of OSN EVs described in greater detail elsewhere herein to the subject in need thereof.

In some embodiments, the subject in need thereof has, has had, and/or is suspected of having a nerve injury, nerve death, aberrant neuron connectivity, aberrant neuron activity, a neuropathy, or any combination thereof. In some embodiments, the subject in need thereof has, has had, and/or is suspected of having a neurodegenerative disease, disorder, and/or condition. In some embodiments, the subject in need thereof has, has had, and/or is suspected of having an epilepsy, a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, and/or is suspected of having a CNS neuron/nerve and/or a peripheral neuron/nerve injury, disease, disorder, and/or condition.

In some embodiments, the subject in need thereof has, has had, and/or is suspected of having an epilepsy, a seizure disease, disorder or condition, or a disease, disorder, or condition in which seizures are a symptom or result of the disease, disorder, or condition, including but not limited to non-epileptic seizures. In some embodiments, the epilepsy, the seizure disease, disorder or condition, or the disease, disorder, or condition in which seizures are a symptom or result of the disease, disorder, or condition is Dravet syndrome, childhood absence epilepsy, gelastic epilepsy, Landau Kleffner syndrome, Lennox-Gastaut syndrome, Doose syndrome (myoclonic astatic epilepsy), West syndrome, benign Rolandic epilepsy, childhood idiopathic occipital epilepsy, juvenile myoclonic epilepsy, early myoclonic encephalopathy, Jeavons Syndrome, Febrile-illness related epilepsy syndrome, Ohtahara syndrome, panayiotopoulos syndrome, temporal lobe epilepsy, Rett Syndrome, CDKL5 disease, stroke, brain tumor, cardiovascular disease or disorder, drug toxicity or withdrawal, psychogenic disorder, fevers, brain trauma, PCDH19 GCE epilepsy, and/or the like, abdominal epilepsy, and/or any combinations thereof.

In some embodiments, the subject in need thereof has, has had, or is suspected of having a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof. In some embodiments, the subject in need thereof has, has had, or is suspected of having a CNS neuron/nerve and/or a peripheral neuron/nerve injury, disease, disorder, and/or condition. In some embodiments, the disease, disorder, and/or condition is a genetic disease, disorder, and/or condition. In some embodiments, the disease, disorder, and/or condition is not a genetic disease, disorder, and/or condition.

Administration can be hourly, daily, weekly, monthly, or yearly. Administration can be one or more times an hour, one or more times a day, one or more times a week, one or more times a month, or one or more times a year. Administration can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more times as appropriate per hour, day, week, month and/or, year.

In some embodiments, the composition containing the OSN EVs described elsewhere herein can be administered along with a co-therapy. The co-therapy can be administered in the same formulation and/or be contained in the OSN EVs (e.g., as a cargo), at substantially the same time, or sequentially to the composition and/or formulation containing the OSN EVs. In some embodiments, the agent(s) in addition to the OSN EVs, co-therapy can treat and or prevent a disease or symptom thereof. In some embodiments the co-therapy is optionally a polynucleotide, a polypeptide, a nutrient (e.g., lipid, amino acid, carbohydrate, peptide, protein, sugar, vitamin, mineral, and/or the like), genetic modifying system or component thereof, antibody or fragment thereof, aptamer, affibody, small molecule chemical agent (e.g., a therapeutic and/or prevention), an immunomodulator, a hormone, an antipyretic, an anxiolytic, an antipsychotic, an analgesic, an antispasmodic, an anti-inflammatory agent, an anti-epileptic agent, an anti-histamine, a growth factor, an anti-infective, a radiation sensitizer, a chemotherapeutic, a neurotransmitter antagonist, a neurotransmitter agonist, or any combination thereof.

Administration of the OSN EV composition and/or any co-therapy can be by any suitable administration routes and/or methods. Exemplary suitable administration routes and/or methods are described in greater detail elsewhere herein.

As previously described, in some embodiments the olfactory derived EVs are patterned and optionally dried on the surface of a device. In some embodiments, a method of treating a nerve injury or promoting nerve growth includes implanting a device comprising olfactory derived EVs described herein patterned on one or more surfaces of the device. In some embodiments, the patterns are in a design to promote correct or desired neuron growth.

In some embodiments, the olfactory neuron derived EVs of the present disclosure can be used in vitro, in vivo, or ex vivo to promote stem cell division. In some embodiments, the stem cells are pluripotent stem cells. In some embodiments, the stem cells are totipotent stem cells. In some embodiments, the stem cells are multi-potent stem cells. In some embodiments, the stem cells are olfactory epithelium stem cells. In some embodiments, the stem cells are olfactory neuron stem cells. In some embodiments, the stem cells are epithelial stem cells.

In some embodiments, the olfactory neuron derived EVs of the present disclosure are used in vitro, in vivo, or ex vivo to promote cell reprogramming. In some embodiments, the olfactory neuron derived EVs of the present disclosure can be used to reprogram cells to produce induced pluripotent stem cells. In some embodiments, differentiated or somatic cells are cultured in the presence of olfactory with olfactory neuron derived EVs of the present disclosure.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Olfactory Sensory Neuron Derived EVs

Peripheral neuropathy involves damage to neurons in the peripheral nervous system (PNS), and has numerous causes, including: traumatic injury, infection, metabolic disorder, exposure to environmental toxins, genetic causes, and others. For example, injury can be caused by damage during surgery, vehicular collision, or prolonged immobility. Recovery time depends on the extent of damage, and regeneration can be as slow as 1-2 mm per day. Critically, there is no treatment to accelerate this regrowth (1,2).

Current approaches to repair neuronal damage include microsurgery, glues to promote regrowth, and the development of conduits to bridge the proximal and distal stump (3,4). Another approach is to use cell-based therapies. In particular, the use of Schwann cells (a form of glia) is the most commonly studied such therapy (5). Schwann cells enwrap axons, providing neurotrophic factors to support axon regrowth, myelin to protect and insulate axons, and modulate the immune response. However, they proliferate slowly and are difficult to obtain. Alternatives have included stem cells isolated from various sources, which are then differentiated into Schwann-cell like cells.

More recently, efforts aimed at identifying the factors produced by Schwann cells to promote regrowth suggest that extracellular vesicles (EVs) are important for this process. EVs are secreted by all cell-types, and contain RNAs, DNA, and protein. They have recently been identified as an important form of cell-cell communication. Uptake of EVs by cells can lead to significant changes in cellular homeostasis.

Schwann cell derived EVs were shown to promote the regrowth of peripheral neurons of neurons grown in culture and in rodents with sciatic nerve injury (6). Other groups have also shown that EVs derived from olfactory ensheathing cells (another cell-type similar to Schwann cells and are also glial-like) also can promote regrowth of neurons in culture and in surgical models (7). These results show glial culture-derived EVs are an important mechanism for promoting axonal regrowth both in culture and in vivo.

As noted above, the role of Schwann cells in promoting axonal regrowth has been extensively studied, and EVs derived from Schwann cells promote regrowth. In contrast, the role of neuron culture-derived EVs in axon growth has only been studied in a single paper (8). In this study, cortical neuron culture-derived EVs inhibited growth of neurons grown in culture, and did not enhance regrowth. Further, as far as we are aware, there are no papers on neuronal-derived EVs on regeneration in vivo.

A unique feature of this disclosure is the use of olfactory neuron culture-derived EVs to promote axon growth. As noted above, almost all efforts on promoting axonal growth have focused on glia, and glial-derived EVs. This is based on decades of study showing the importance of glia in promoting neuronal survival and function. However, a major area of research that surprisingly has not been studied is the role of the neurons themselves in this process. In particular, we focused on olfactory neurons because they are well-known to regenerate constantly throughout the life of the animal.

Applicant theorized that this regrowth confers unusual properties to olfactory neurons as compared with cortical neurons (which do not regenerate). Applicant therefore isolated EVs from cultured olfactory neurons. The initial results show these neuronal culture-derived EVs, in contrast to the prior study on cortical neurons, do in fact promote neuronal regrowth. Applicant demonstrated this using two model systems, applying olfactory neuron culture-derived EVs to cultures of olfactory neurons (to assess the effects of adding additional EVs to olfactory neurons), and also to cultures of dorsal root ganglia (DRG, a commonly studied peripheral neuronal type but clearly distinct from olfactory neurons). For example, in FIG. 1A-1B, Applicant quantitated neurite length of DRGs grown in culture with and without the addition of olfactory neuron culture-derived EVs. The EVs clearly increase the number of DRGs with increased neurite length, demonstrating an effect on DRG growth. Thus, olfactory neuron culture-derived EVs can enhance the growth not only of olfactory neurons, but also of a distinct neuronal cell-type.

### Purification of Olfactory EVs

Purification of EVs followed published procedures. Briefly, EV purification involved dissecting olfactory epithelia from mice. Mice were of varying age, but typically were young postnatal animals (postnatal day 6 to 10). Epithelia recovered were enzymatically digested and then plated on coverslips coated with an appropriate adhesive, such as poly ornithine. An appropriate neuronal culture media (typically BrainPhys supplemented with SM1 and G418) was then added and neurons are cultured for about 2 days. The supernatant from multiple samples is collected and pooled. EVs are isolated by using a well-established centrifugation protocol involving sequential low-speed spins followed by a high speed (100,000×g) to pellet EVs. EVs were resuspended in PBS and frozen prior to use.

The EVs can be isolated from olfactory neurons isolated from different ages of the subject's life. Without being bound by theory, it is believed embryonic EVs may be better than adult EVs at promoting growth, or adult EVs might be better because they are derived from regenerating neurons.

The olfactory neuron culture-derived EVs can be used to help enhance peripheral neuron regrowth in both humans and animals. They can be incorporated into hydrogels, used during surgery to promote repair, injected systemically to enhance growth, and could be inhaled as well. It is also possible, because olfactory neurons cross into the central nervous system, that they may also enhance regeneration of central nervous system neurons. Thus, they could also promote regeneration of neurons affected by degenerative diseases such as Alzheimer's and Parkinson's. Finally, because EVs are potential vehicles for drug delivery, studies have been performed showing EVs derived from human HEK293 cells do not appear to induce an immune response when injected into mice. Thus, it is possible that EVs from olfactory neurons in mice (which can be generated rapidly) can be applied to other species.

### References Related to Example 1

1. Pfister B J, Gordon T, Loverde J R, et al. Biomedical engineering strategies for peripheral nerve repair: Surgical applications, state of the art, and future challenges. Critical Reviews in Biomedical Engineering. 2011; 39(2):81-124.
2. Takahashi K., Yamanaka S. Induction of pluripotent stem cells from mouse embryonic and adult fibroblast cultures by defined factors. Cell. 2006; 126:663-676.
3. Xue, W., Shi, W., Kong, Y., Kuss, M., and Duan, B. Anisotropic scaffolds for peripheral nerve and spinal cord regeneration. Bioact. Mater., 2021, 6(11): 4141-60.
4. https://www.intechopen.com/chapters/55127.
5. Hood, B., Levene, H. B., Levi, A. D. Transplantation of autologous Schwann cells for the repair of segmental peripheral nerve defects, Neurosurg. Focus, 2009 26:1-9.
6. Lopez-Verilli, M. A., Picou, F., Court, F. A. Schwann cell-derived exosomes enhance axonal regeneration in the peripheral nervous system Glia, 2013. 61(11):1795-806.
7. Xia, B, Gao, J. Li, S. et al. Extracellular Vesicles Derived From Olfactory Ensheathing Cells Promote Peripheral Nerve Regeneration in Rats. Front. Cell. eurosci., 2019, 13, Article 548.
8. Gong, J., Korner, R., Gaitanos, L., Klein, R. Exosomes mediate cell contact-independent ephrin-Eph signaling during axon guidance. Journal of Cell Biol., 2016, 214(1): 35-44.

### Example 2—Effects of Extracellular Vesicles on Neuronal Regeneration in the Olfactory System

Canine spinal cord injuries are 2% of cases presented to veterinarians. Acute spinal cord injury has a poor prognosis and surgery must occur within 24 hours of injury but is highly invasive and variably successful. A less invasive method that has proven successful is the injection of olfactory ensheathing cells into the injured spines of dogs. How olfactory ensheathing cells, a type of glial cell, produce this effect is not currently understood. The olfactory nervous system is the most exposed group of neurons to the exterior environment leading to the most frequent damage and the necessity of regeneration. While adult neurons in the CNS and parts of the PNS have limited capability to regenerate post damage, olfactory sensory neurons (OSN) regenerate every ˜30 days. Further, past studies have preliminarily found that Schwann cell derived extracellular vesicles (EVs) injected in vivo improved the axonal regrowth of mechanically damaged dorsal root ganglia. EVs, which are responsible for intercellular communication, are created by exocytosis from the lipid bilayer and can contain cytosolic proteins, membrane proteins, mRNAs, noncoding RNAs, and even DNA. Within the olfactory system, EVs are produced by OSN and surrounding glial cells, keeping the two cell types in communication. We hypothesized that OSN EVs would have an effect on the rate and accuracy of neuronal regeneration in damaged olfactory systems in vivo. OSN were destroyed using methimazole (IP), a compound that induces the degeneration of the OSN in the epithelia while maintaining the integrity of the lamina propria and cribriform plate. EV injections were tested at 2, 4, and 8 μg dosages, and mice OSN regrowth was examined at days 0, 14, 28, and 42.

### Example 3—Determining the Effect of Neuron-Derived Extracellular Vesicles on Axonal Regeneration of Dorsal Root Ganglia

Peripheral nerve diseases such as traumatic neuropathies and sensory neuropathies have been found in dogs and cats. A problem in this field is finding treatments for nerves that can slow the progress of these diseases. Nerve growth factor has been shown to promote the initial sprouting of axons, but other factors that help guide axons to their appropriate targets are still poorly characterized. Studies with Schwann- and olfactory ensheathing cell-derived extracellular vesicles (EVs) have been shown to promote axonal regeneration after nerve damage. This study tested whether other neuron-derived EVs also promote the growth of dorsal root ganglia (DRG) axons. This was accomplished by isolating individual DRG neurons from mice and culturing them with or without neuron-derived EVs. Additionally, whole DRGs were plated and treated with or without EVs. The DRG neurons and whole DRGs were stained with beta-tubulin antibody to detect neurite growth, and then analyzed by comparing axonal length between EV treated and control conditions. The application of neuron-derived EVs may provide a starting point for developing a treatment that can delay the progression of peripheral nerve diseases.

### Example 4—Regeneration in the Mouse Peripheral Nervous System

There are approximately 12,500 new cases of spinal cord injuries per year. 38 percent are caused by vehicular crashes and about 30 percent are due to falls. Approximately 340,000 currently living with a spinal cord injury. The average lifetime costs associated directly or indirectly with a spinal cord injury range from about $428,000 to $1.4 million, based on severity.

Olfactory neurons regenerate constantly through life with neurons being born approximately every 30 days. Newly born olfactory neurons must find their way back to their proper targets in the olfactory bulb in the brain. Granger et al., 2012 demonstrated spinal cord regeneration in dogs in a randomized controlled trial at Cambridge Veterinary Hospital. To do this they removed a special type of cell called the olfactory ensheathing cell (OEC), a type of glial cell, from the nasal passageways of the dogs, grew them in culture until a sufficient number had been produced, and then transplanted them at the site of injury. Tabakow et al. (2014, Cell Transplant., 23, 1631-1655) demonstrated functional regeneration of supraspinal connections in a patient with a transected spinal cord following transplantation of bulbar olfactory ensheathing cells. Applicant assessed whether the regenerative properties of the olfactory system neurons can be used to repair damage in the CNS and/or PNS.

Extracellular vesicles, including exosomes and microvesicles, are small, nano-to-micrometer vesicles that are released from cells.

Paolicelli et al. (Neuroscience, 405(1) (2019) summarized cell-to-cell communication by extracellular vesicles between neurons, astrocytes, and microglia within the central nervous system. See e.g., Paolicelli et al. at FIG. 1. However, there is no understanding to Applicants of effect of olfactory neuron derived EVs in the peripheral nervous system.

Without being bound by theory, Applicant tested two hypotheses: (1) OEG-derived EVs promote OSN regeneration; and (2) OSN-derived EVs do not promote OSN regeneration. Briefly, “control” OSN derived EVs were generated by culturing primary olfactory neurons (FIG. 2A). Neuronal EVs were then purified (FIG. 2B). Purified olfactory neuron EVs were then applied to cultured OSNs. Total outgrowth, longest branch length, and neurites were evaluated (FIG. 2C-2F). Based on the hypotheses made, the results were unexpected. It was observed that OSN-derived EVs do promote OSN regeneration.

To examine the effect of OSN-derived EVs on in vivo regeneration Applicant destroyed the olfactory sensory neuron layer in mice via methimazole on Day 0. Ablation by methimazole forces regeneration of olfactory cells. On days 5, 12, 19, 26, 33, and/or 40 post day 0, OSN-derived EVs were delivered to the mice intranasally at varying amounts. Saline and methimazole (MZ or MI) in saline were delivered as controls. The amount of EVs delivered in a saline carrier were 2 μg, 4 μg, or 8 μg. As shown in FIG. 3, OSNs form a layer within the olfactory epithelium. Sustentacular cells provide various support functions for the olfactory epithelium and neurons.

Applicant evaluated neuronal regeneration for correct and incorrect regeneration to the olfactory bulbs. See FIG. 4A-4B. Mombaerts, Cell. 87(4):675-686 (1996) provided an olfactory sensory map demonstrating correct neural pathways from the nasal epithelium to the olfactory bulb. See e.g., Momaerts. 1996. at e.g., FIG. 2A-2B.

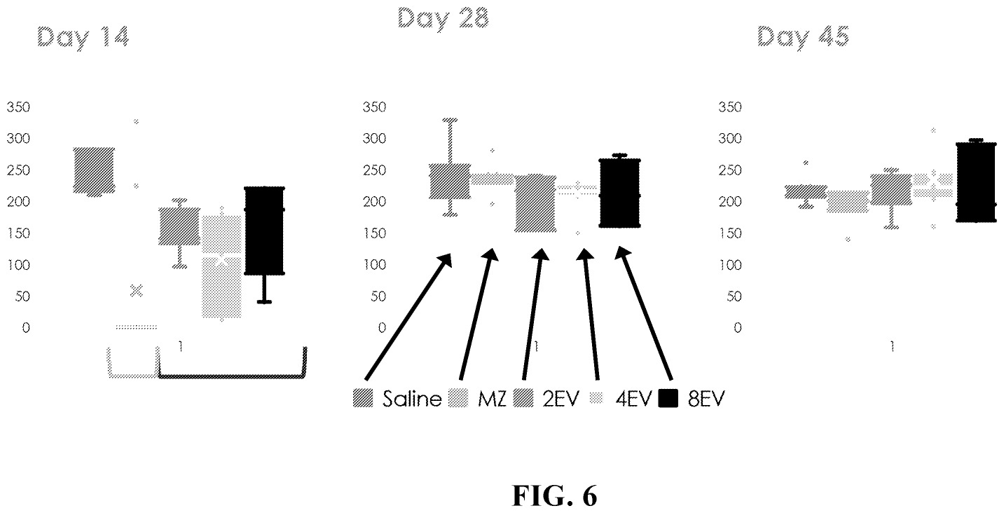

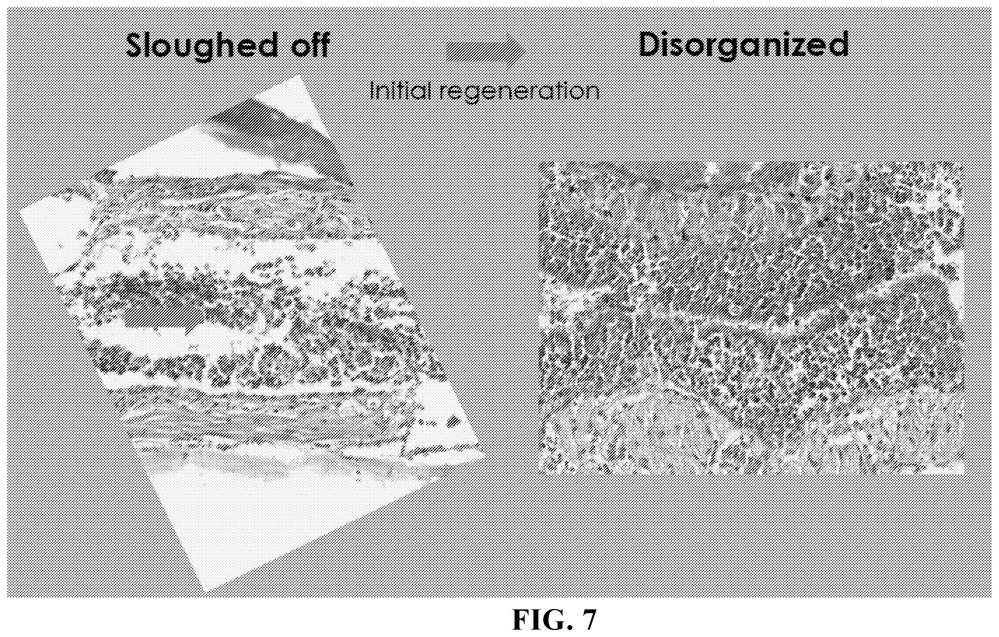

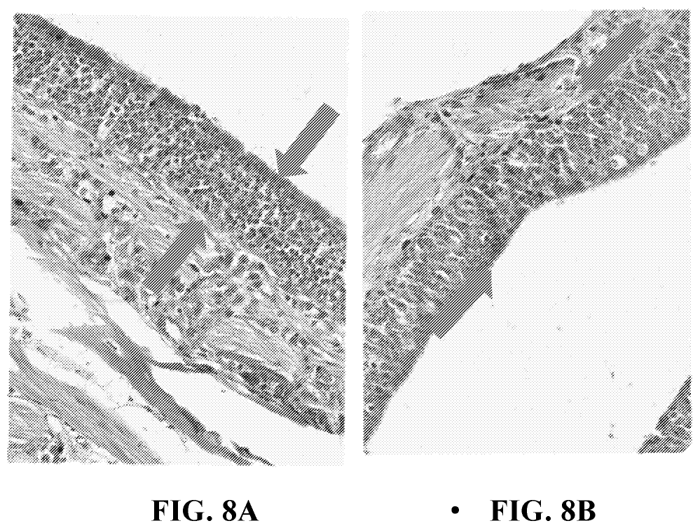

FIG. 5A-5B show fluorescent and stained microscopic images in saline (FIG. 5A) and methimazole ablated (FIG. 5B) olfactory epithelium. As shown in FIG. 6, OSN derived EVs promoted epithelial regeneration. As shown in FIG. 7, methimazole caused sloughing and the initial regeneration was disorganized. FIG. 8 shows the regeneration of the olfactory epithelium and neurons in saline control and EV treated mice.

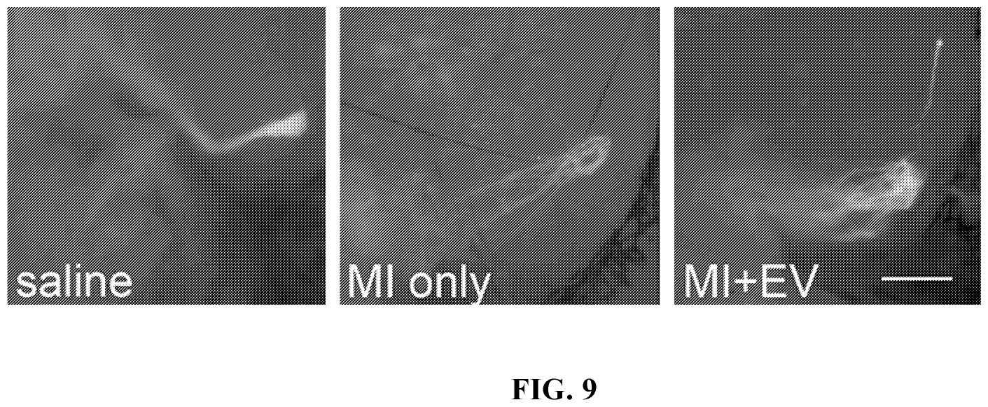

FIG. 9 shows glomeruli in saline control, MI only control, and EV treated groups at day 21 after ablation.

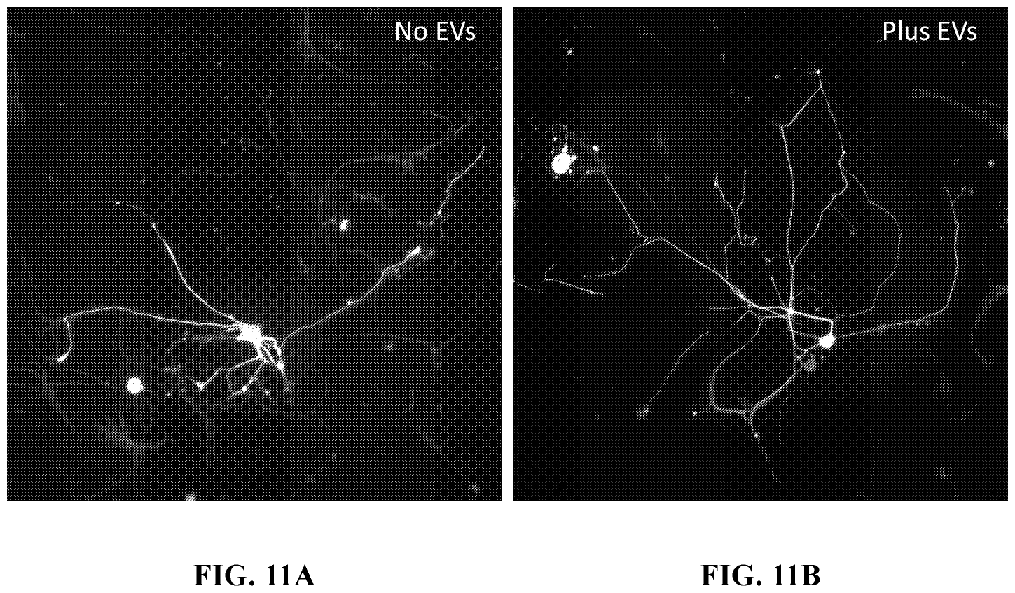

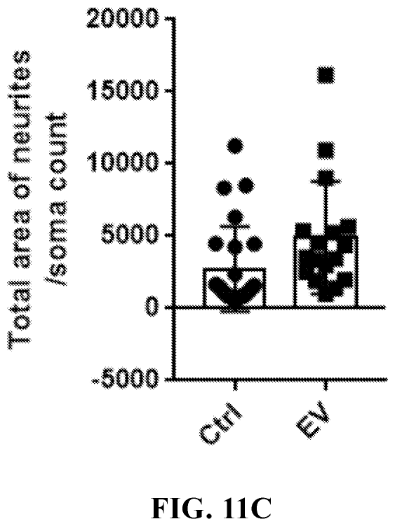

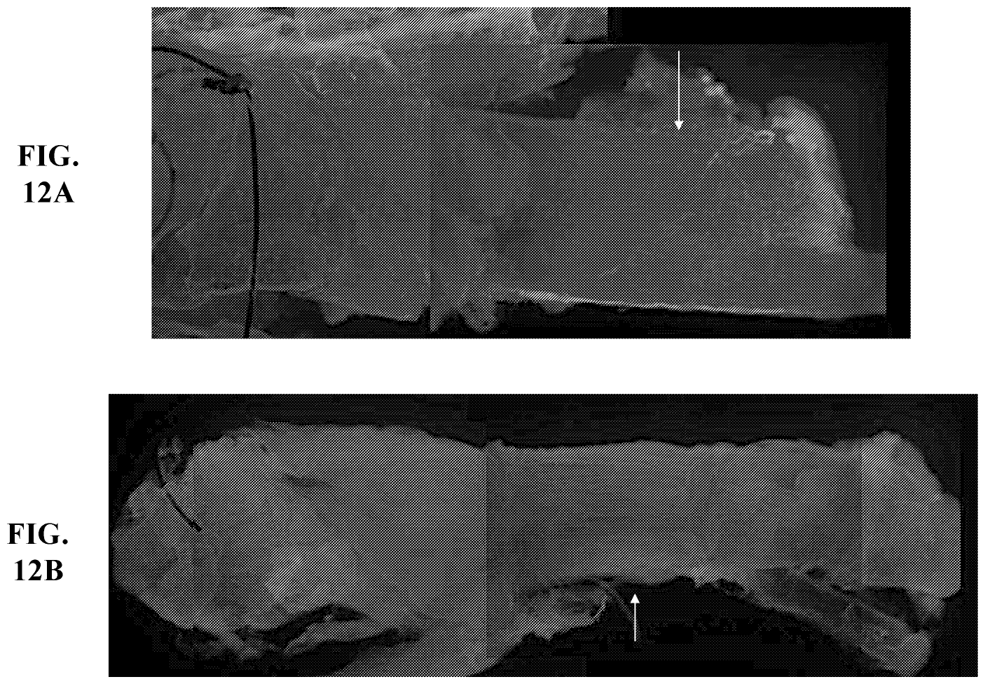

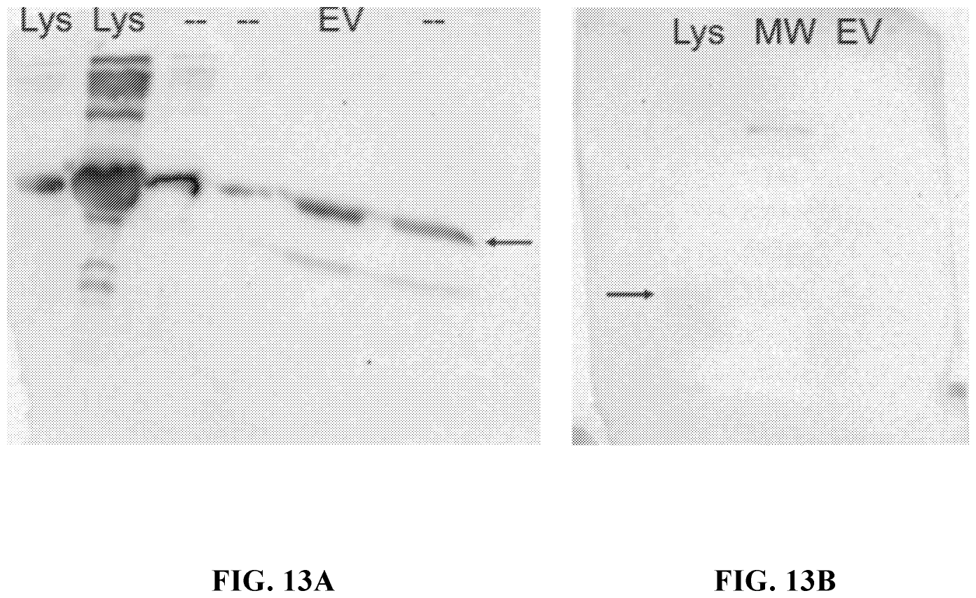

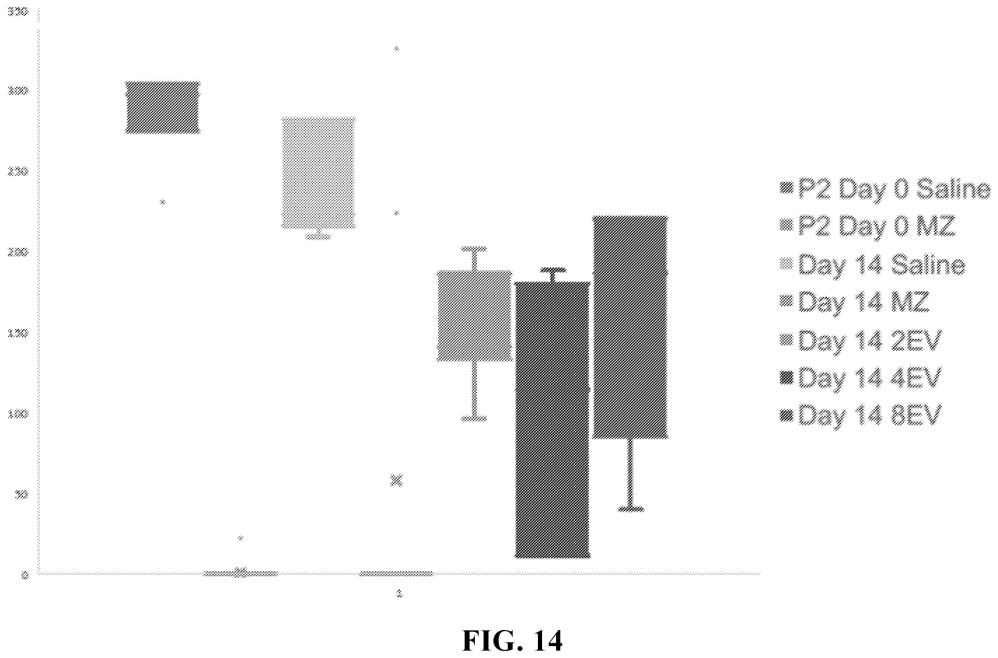

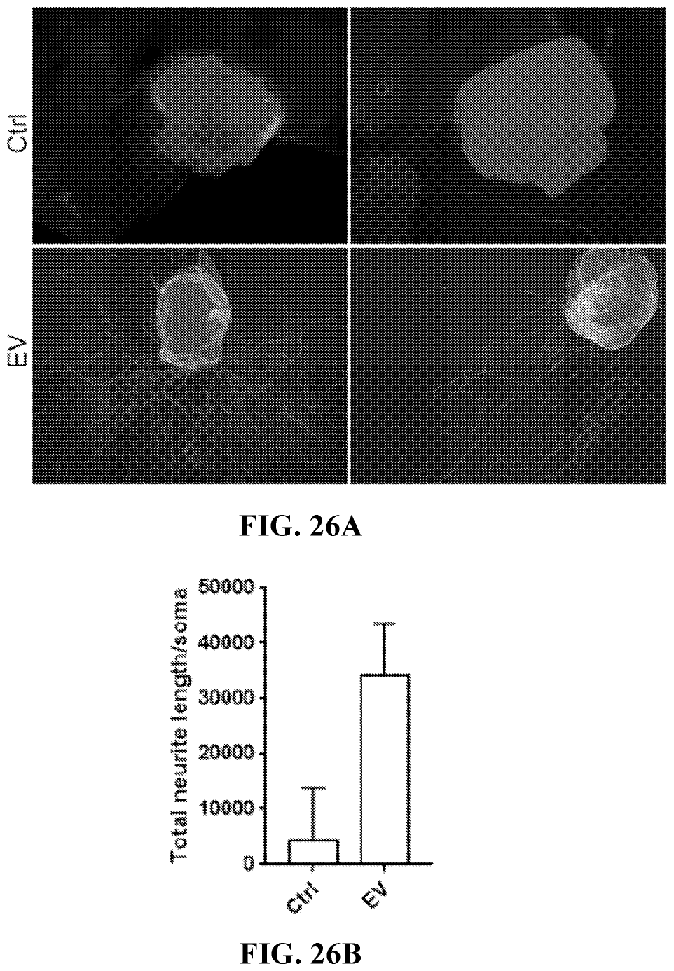

As shown in FIG. 11A-11C EVs from cultured OSNs can increase the total area of neurites in regenerating neurons. Further, in cultured DRGs, treatment with OSN derived EVs increased neurite length (FIG. 26A-26B).

In general, in the epithelial cells at Day 14 post MZ, EV treated group was more improved than the MZ only group. Variable morphologies were observed. Glomeruli were undefined in all groups at Day 14. At Day 28 all conditions were healthy. At day 28 the group treated with 8 μg EVs had the healthiest glomeruli. At Day 45 all conditions were healthy. At day 45 the group all glomeruli were healthy, with the 4 μg and 8 μg EV treated groups having the most defined glomeruli.

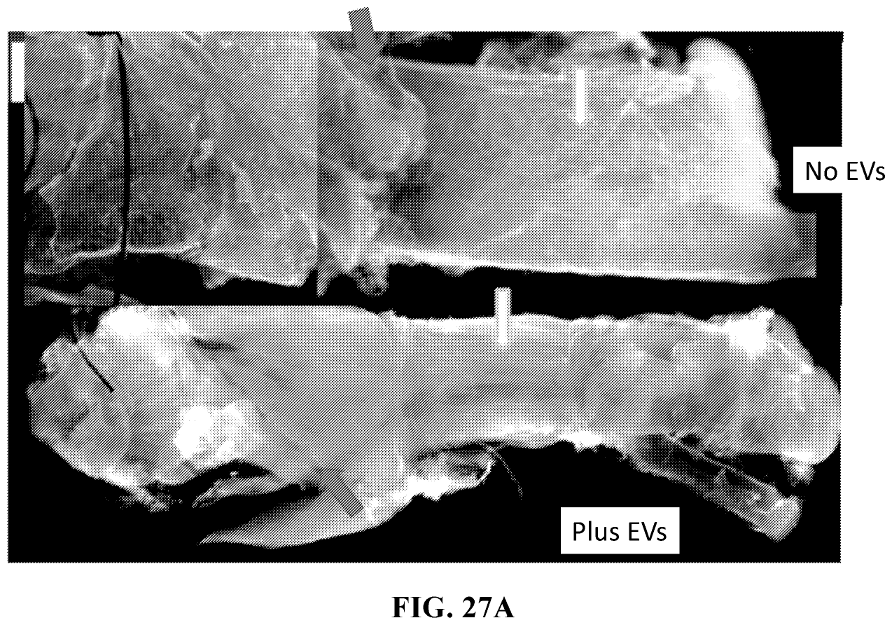

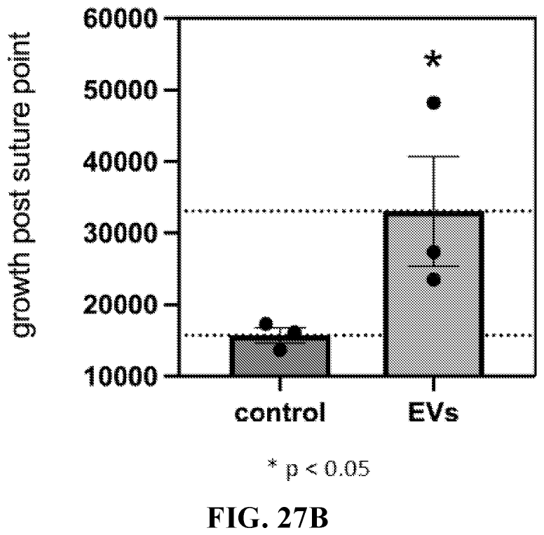

EVs from cultured OSNs were examined for their ability to improve peripheral nerve regrowth after injury. Applicant injured the sciatic nerve according to a protocol described in Niemi et al., 2020. Methods In Molecular Biology Protocols: Axon Degeneration. DOI: 10.1007/978-1-0716-0585-1_16. OSN EVs were delivered to the point of injury and as shown in FIGS. 12A-12B and 27A, treatment OSN EVs improved the organization of the axons, which is indicative of improved neuron regeneration of a peripheral nerve. Further, treatment with EVs increased neuron growth post suture point as compared to the control (FIG. 27B).

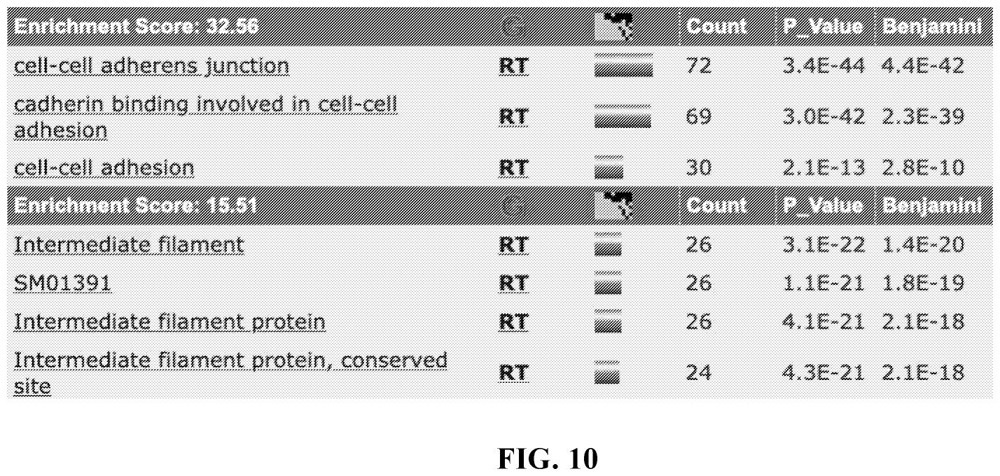

Applicant investigated molecules that promoted OSN regeneration. As shown in FIG. 10, Nano LC MS/MS returned 559 proteins that appeared involved. LC MS/MS results also demonstrated that the signal transduction pathway for delta protocadherins was also over-represented.

Spatial patterns of gene expression in the olfactory bulb have been examined. See e.g., Lin et al., PNAS. 2004. 101 (34) 12718-12723. Williams et al., Front. Neuroscience 2007. Evaluated expression of protocadherins and Bisogni et al., eLife. 2018. evaluated delta protocadherins expression in EVs.

Protocadherin (Pcdh19) is unusual in that it is not a channel protein and that it is X-linked, but only heterozygous females are generally affected. A mutation in Pcdh19 is the cause of female limited epilepsy. There are also comorbidities of hyperactivity. Autism, and obsessive compulsive disorder. Delta protocadherins mediate the promotion of regeneration by OSN-derived EVs.

A CRISPR mouse mutant recreation of known nonsense mutations in Pcdh19 was generated. MRI in humans showed axon tract defects. However, none were identified in mouse models. To date, although learning defects have been observed in mice, no epileptic phenotypes have been observed in mice.

Applicant isolated EVs as previously described (e.g., FIG. 2A-2B) and purified olfactory neuron EVs were then applied to cultured OSNs. Average neurite length was the same between control and protocadherin 19−/− treated mice.

### Example 5—Effects of Extracellular Vesicles (EVs) on Neuronal Regeneration in the Olfactory System

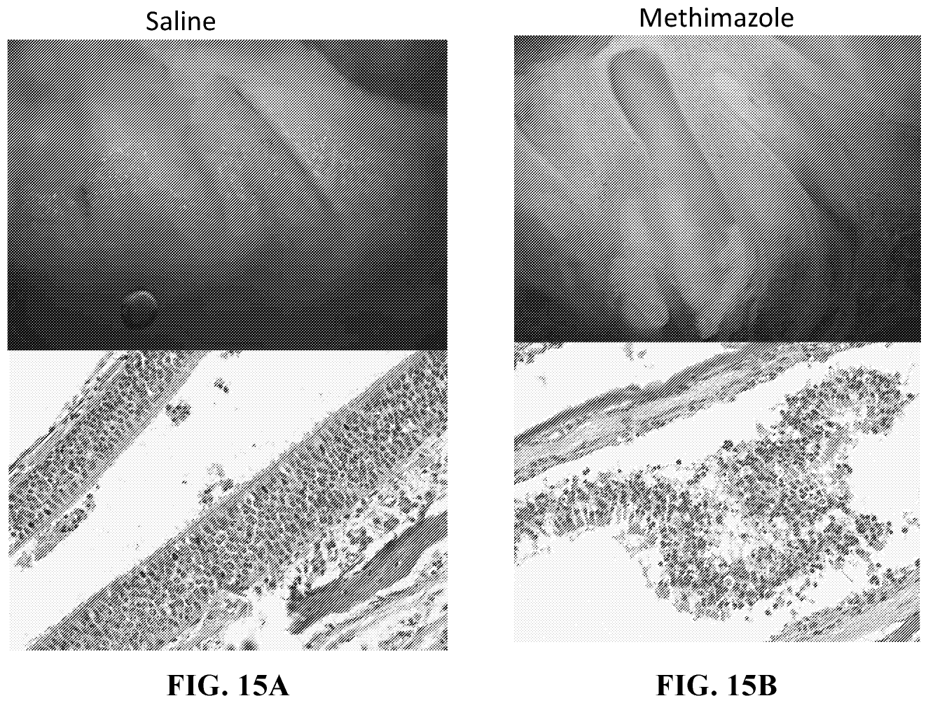

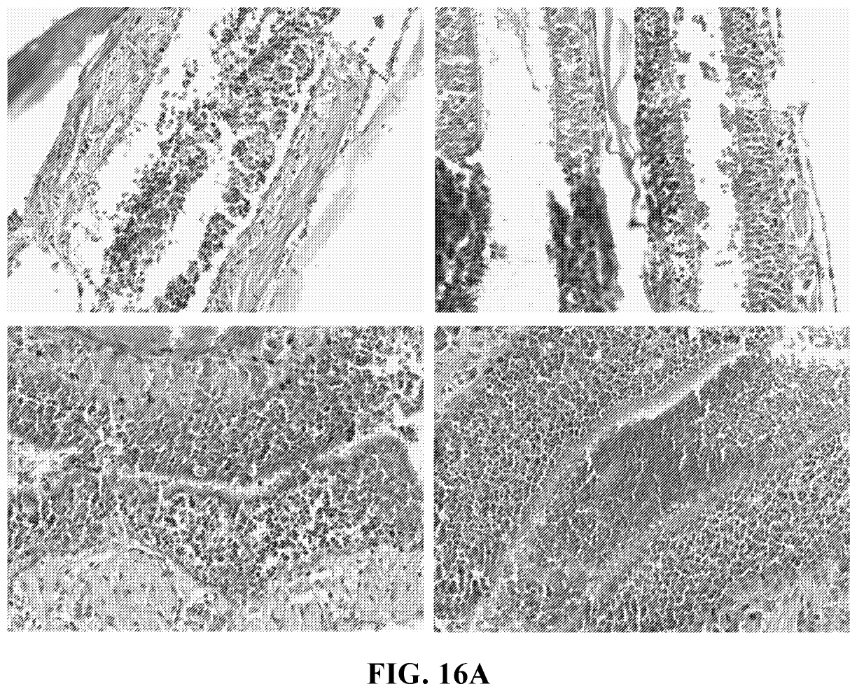

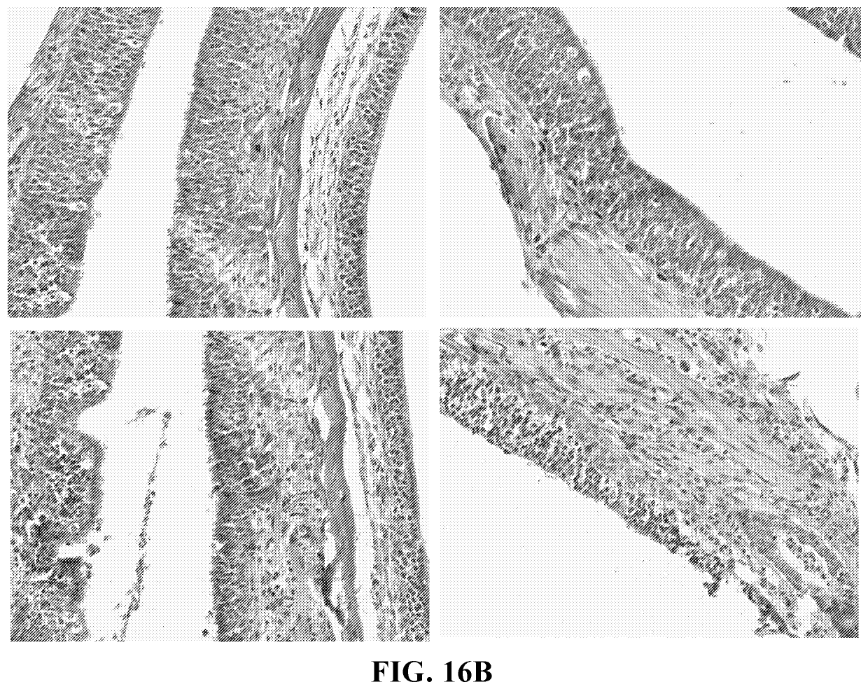

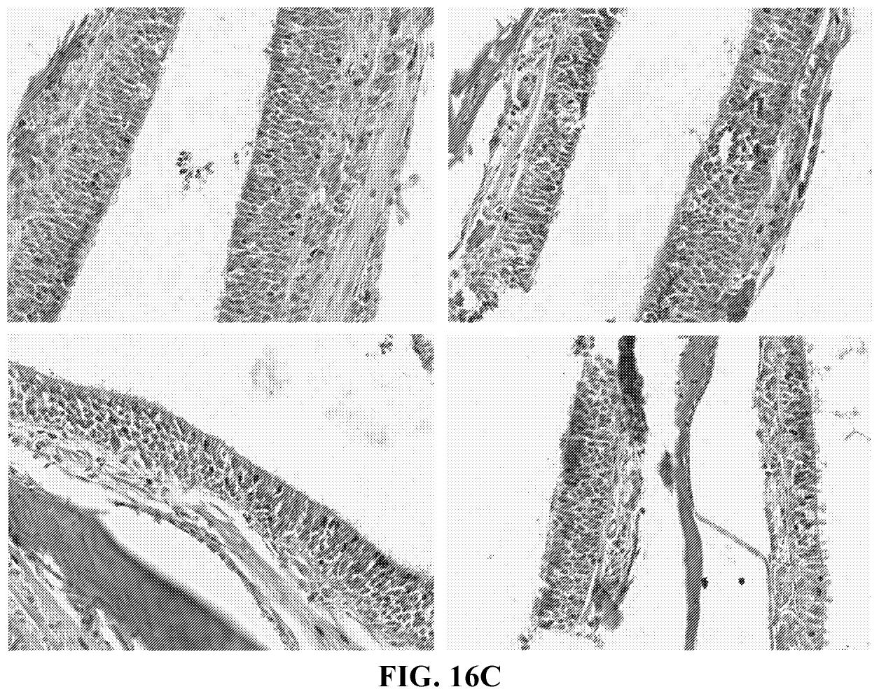

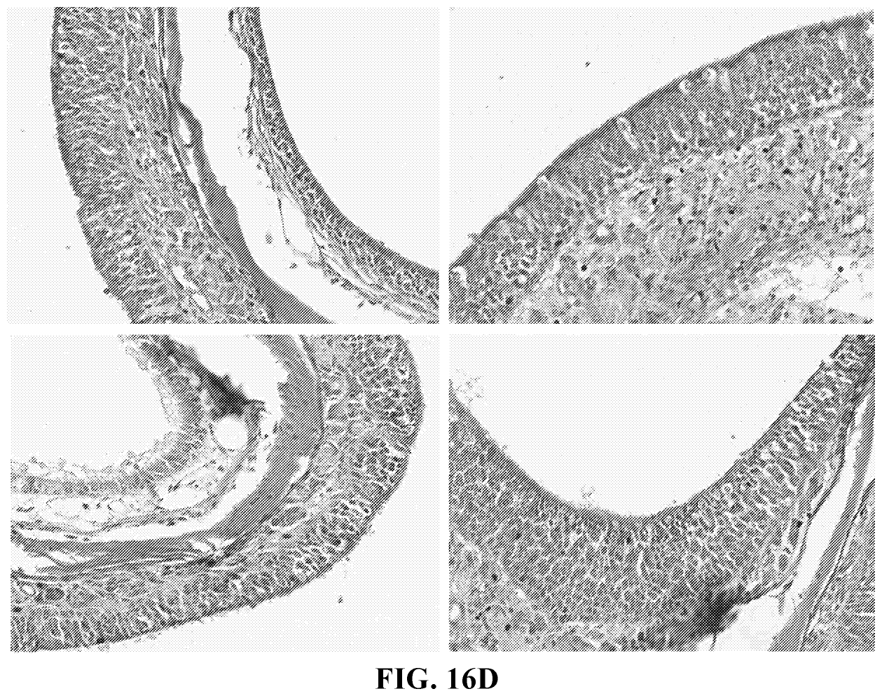

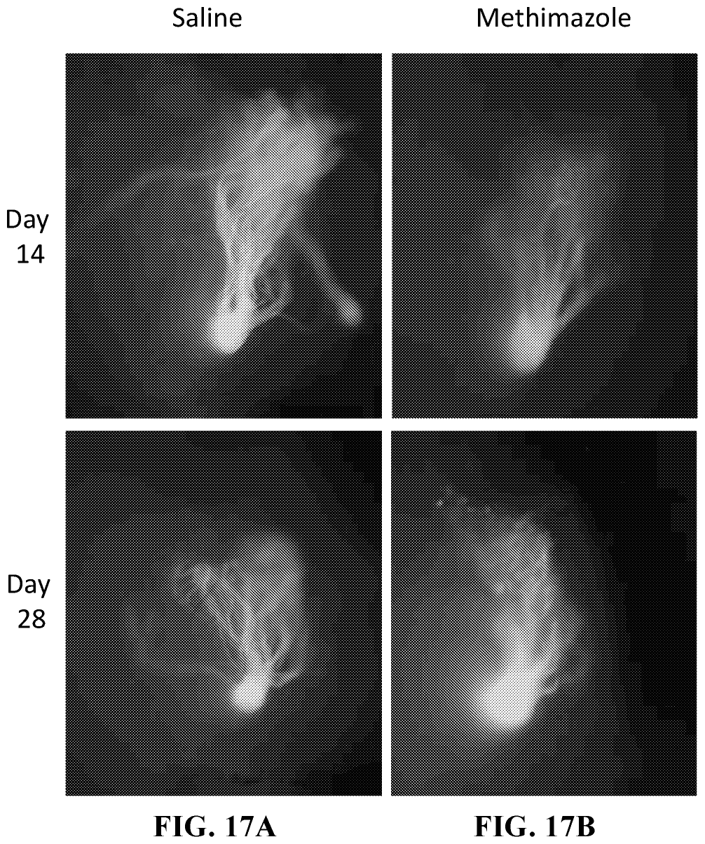

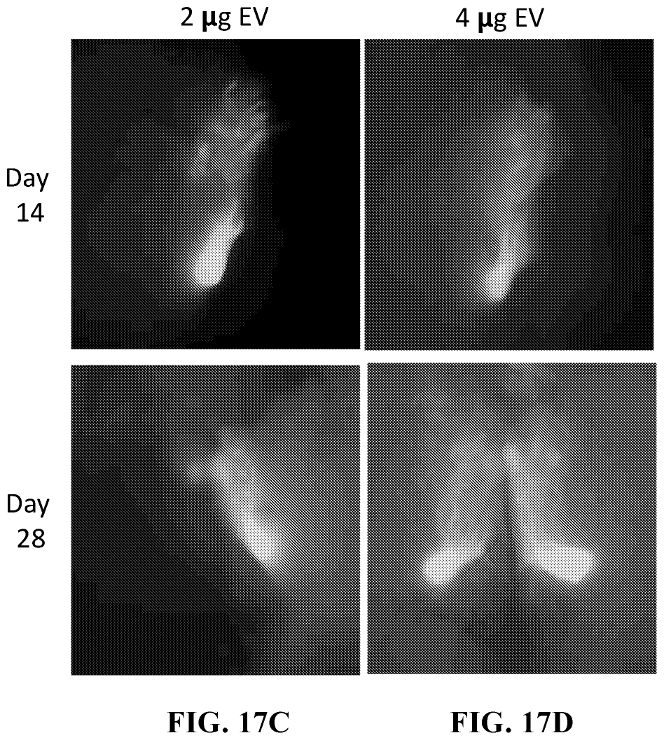

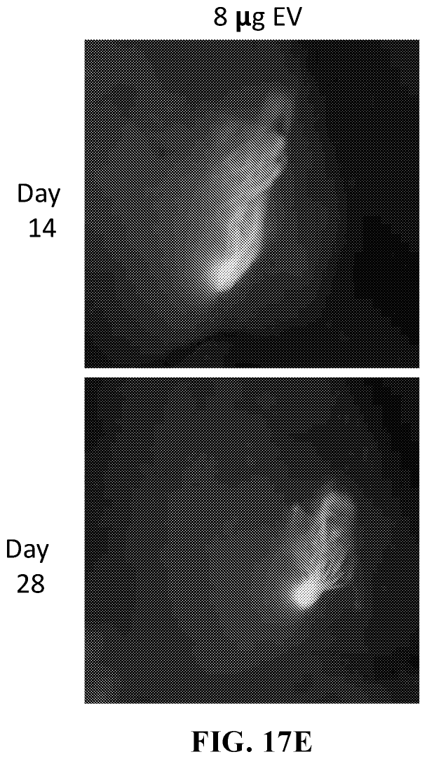

EV stock collected from olfactory sensory neurons was successfully purified as shown in FIG. 25A-25B. The OSN layer was ablated using MZ and EVs were administered to ablated tissue as previously described in Example 4. The OSN layer for each condition's epithelium was measured at five randomized locations and compared. See FIG. 26. FIG. 15A-15B shows fluorescent and stained microscopic images of the olfactory epithelium at Day 0. FIG. 16A-16D show hematoxylin and eosin staining of the olfactory epithelium of the different treatment groups at Day 14. FIG. 17A-17E shows GFP imaging of OSN axons at Day 14 and Day 28 across the treatment groups.

### Example 6—Role of Extracellular Vesicles in Regeneration

Neurodegenerative diseases and conditions are subjects of considerable research due to the difficulties that exist in achieving functional recovery. During embryonic neurogenesis, the developing neuron can form interconnected and strong networks; however, in adulthood, central nervous system (CNS) neurons lose their ability to self-repair and regenerate. The elucidation of novel proteins and mRNA responsible for neuroregeneration is crucial for developing treatments that target neurodegenerative diseases. The peripheral nervous system (PNS) repairs and regenerates throughout life, and thus is a key model for investigating this. Olfactory sensory neurons (OSNs), especially, represent a promising avenue for studying regeneration due to their remarkable regenerative capabilities, possessing the ability to not only extend growth and development but also to continuously regenerate and reestablish connections with the olfactory bulb regardless of injury. These results in the OSNs of mice suggest that OSN-derived extracellular vesicles (EVs) promote neuronal growth. Moreover, Applicant characterized the effects on regeneration from different subtypes of EVs. Finally, Applicant validated a method for manipulating EV content and identified specific proteins that may be important for growth and regeneration. The discovery of important regenerative-inducing factors in the brain will ultimately open new therapeutic avenues for the intervention of neurodegenerative diseases.

### Methods

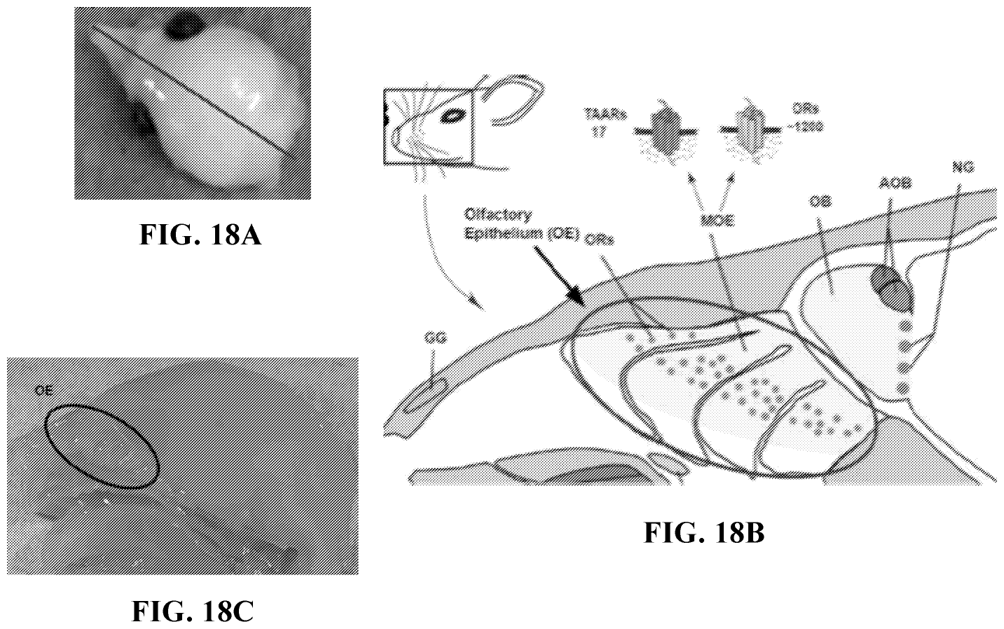

As shown in FIG. 18A-18C, the olfactory epithelium in mice was dissected and isolated. FIG. 18A shows a dorsal view of a neonatal mouse head; line depicts the intended cut through the midsagittal plane. FIG. 18B shows a schematic representation of the mouse olfactory system. FIG. 18C shows a sagittal view of an opened nasal cavity.

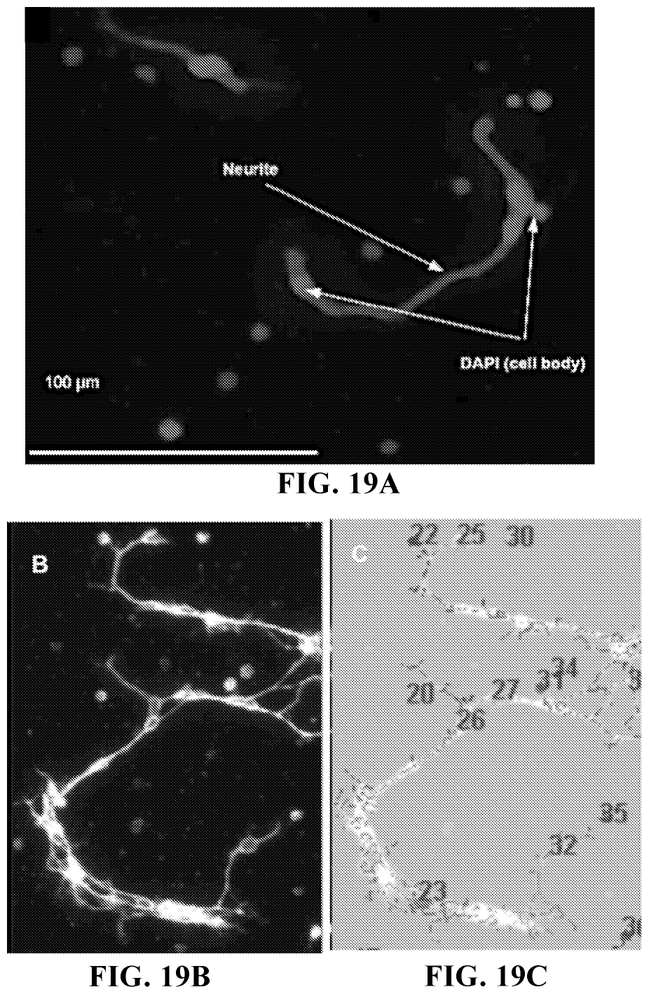

FIG. 19A-19C shows the data analysis apparatus of immunostained sample images of Neurons and ImageJ software for automated analysis of neurons. Applicant analyzed immunostained images (FIG. 19A) via ImageJ software which automatically traces neurons based on interpretation of where cell bodies and neurites are located. (FIG. 19B) shows the input and (FIG. 19C) shows the output, which ultimately converts into an excel sheet for statistical analysis.

### Results

OSN-Derived EVs were Successfully Purified and Validated

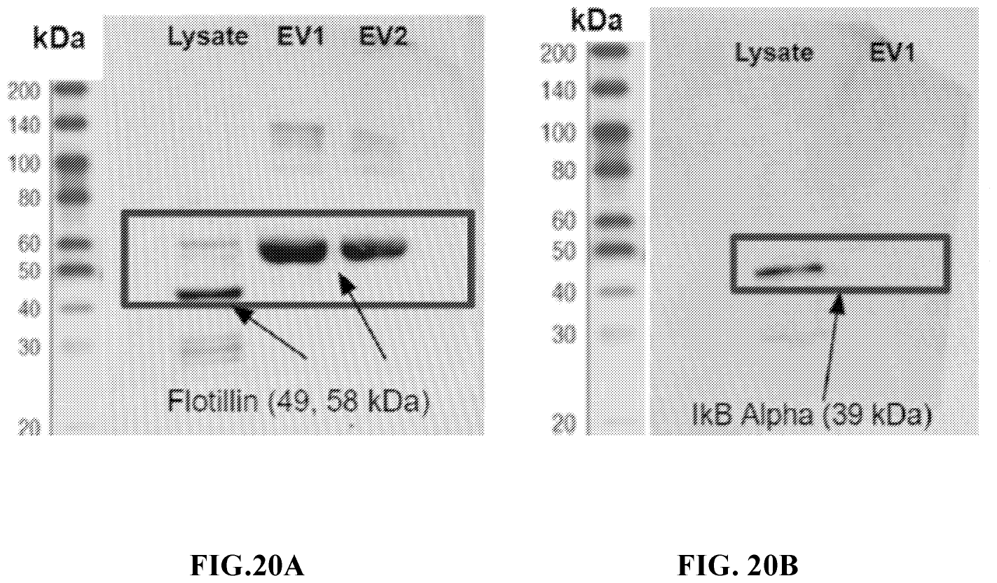

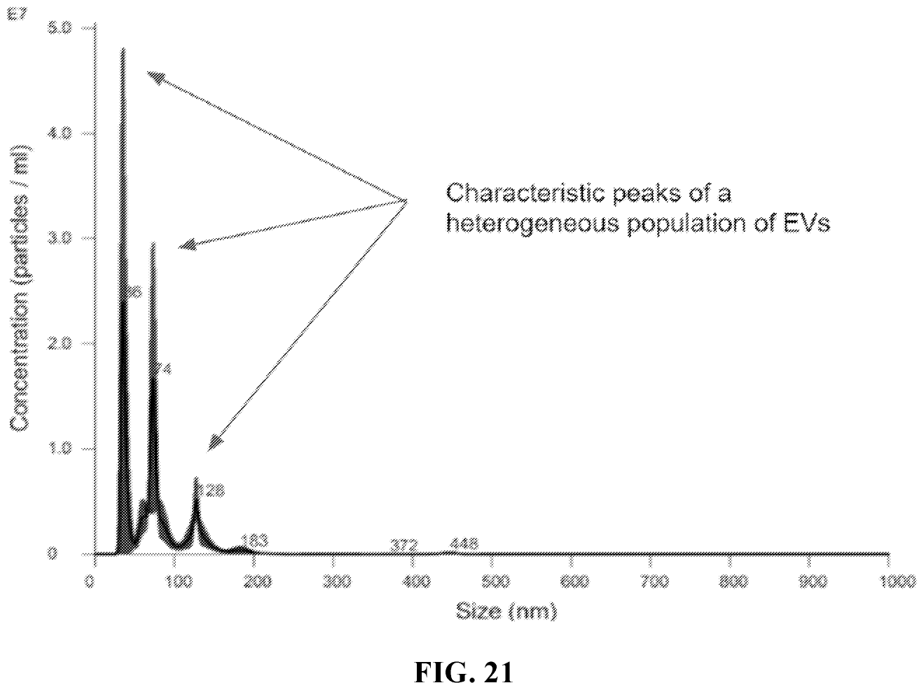

FIG. 20A-FIG. 20B shows a representative western blot for verification of EV Purification. FIG. 20A shows a western blot that demonstrates exposure of lysate and two EV samples to flotillin-2 primary antibody. FIG. 20B shows a western blot that shows exposure of lysate and one EV sample to IkB alpha primary antibody. FIG. 21 shows Nanosight NS300 nanoparticle analysis for secondary verification of EV purification. There are characteristic peaks (36 nm, 74 nm, and 128 nm) as well as smaller ones (183 nm, 372 nm, and 448 nm) that are all within expected ranges in a heterogenous population of EVs (both exosomes and microvesicles).

Overexpression of OSN-derived EVs Significantly Promote Growth in OSN Cultures in vitro

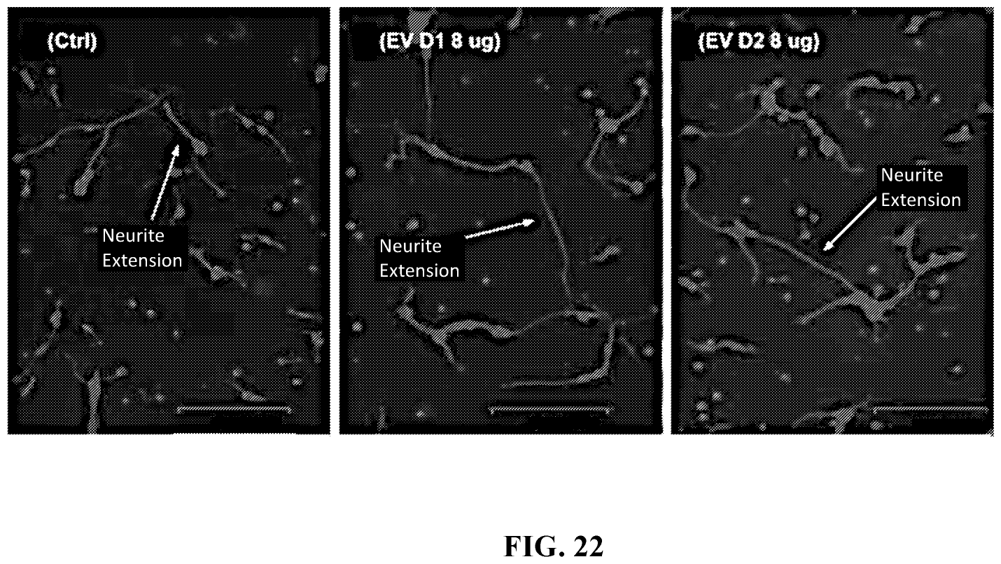

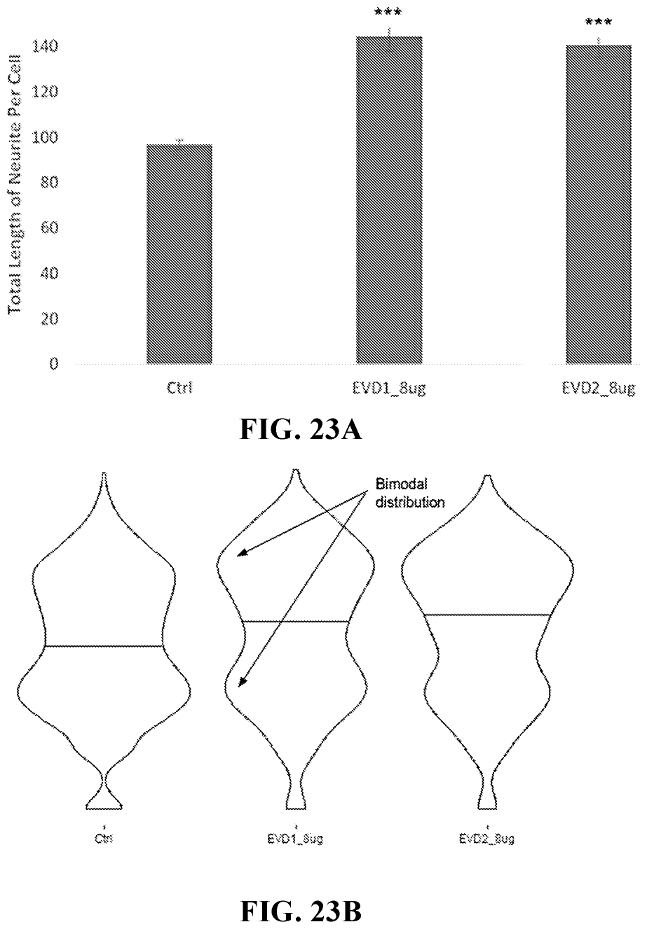

FIG. 22 shows immunostained images of single concentration (8 μg) comparing EVD1 and EVD2 with Ctrl. Neurites are traced in red (as represented in greyscale) and cell bodies are stained with DAPI (blue, as represented as greyscale). Differences (arrows pointing out neurite extensions) can be observed between the two EV types with control with apparent greater growth of neurite lengths. FIG. 23A-23B shows a graphical representation of growth distribution as a measure of total length of neurite per cell. At a selected concentration of 8 μg, both EVD1 and EVD2 showed significantly greater average total length of neurite compared to control (p<<<0.001); no significant differences were observed between the two EV populations (p=0.57). To better visualize the distribution of data, a violin plot (log of average total length of neurite) was made and a bimodal distribution was observed, albeit more prominent in the EV populations than the control. The line represents the mean.

### Isolation of Microvesicle-Only EVs Show Differences in Contributions to Growth

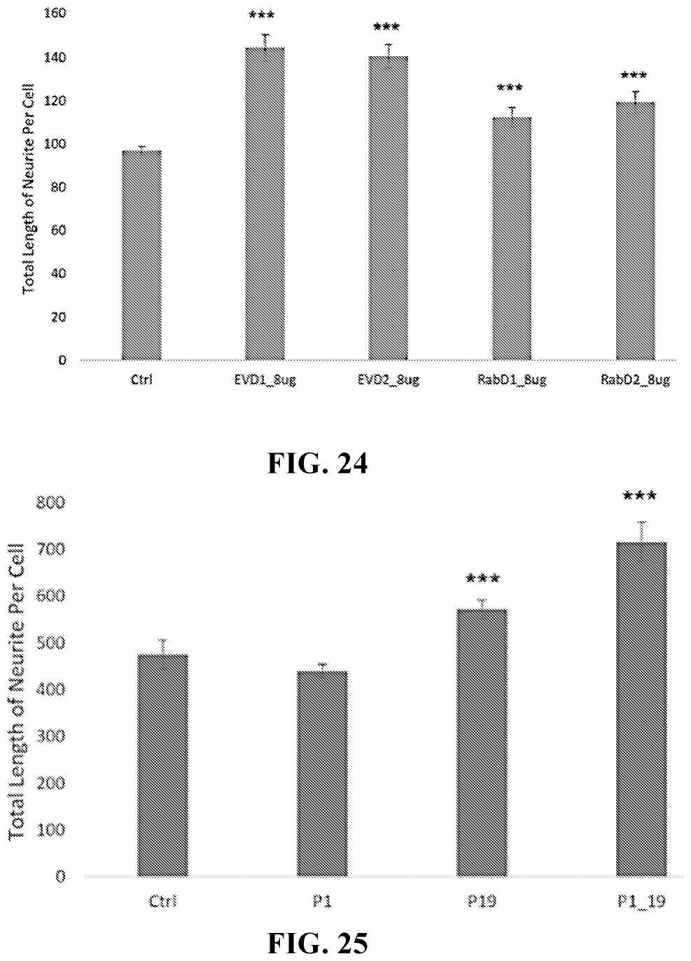

FIG. 24 shows a comparison of Rab27DKO (Exosome-Deficient EVs) with normal EVs. All EV samples (normal and Rab27DKO) were significantly different than control for average total length of neurite (p<<<0.001). EVD1 and EVD2 were also significantly different than RabD1 and RabD1 (p<<0.01). There are no significant differences between EVD1 and EVD2 (p=0.56); similarly, no significant differences were noted between RabD1 and RabD2 (p=0.26).

### Genetic Manipulation of Protocadherin Proteins in EVs Increases Growth

FIG. 25 shows the effects of electroporated Pcdh on Growth in OSNs. Note: ‘P’ was used as a shorthanded abbreviation for Pcdh and P1_19 is the combined Pcdh1 and Pcdh19. There were significant differences in average neurite length compared to control for electroporated Pcdh19 and electroporated Pcdh1+19. No statistically significant differences, however, were observed for Pcdh1 compared to control (p=0.32).

### Discussion

There was a growth-promoting role conveyed by EVs expressed in neuronal cultures. The effect seems to be standardized for EVs extracted 1-day post-culture and 2-days post-culture. This is the first-time data from OSN-EVs has ever been seen in the neuroregenerative field and indicates a clear need to further investigate the roles for neuronal-EVs in growth. Microvesicles produced part of the growth phenotype observed in vitro. Applicant successfully manipulated EV content via electroporation. A key consideration should be made for protein-protein interaction for regeneration.

### Example 7—EV Stamped Devices

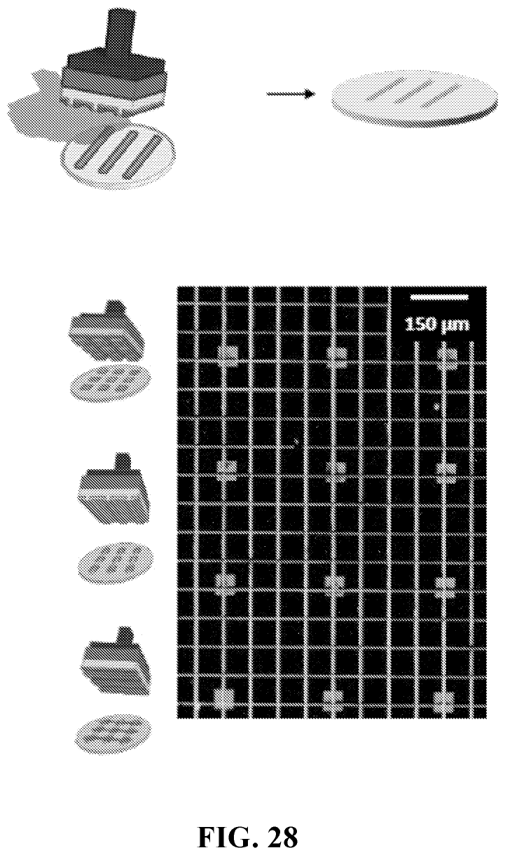

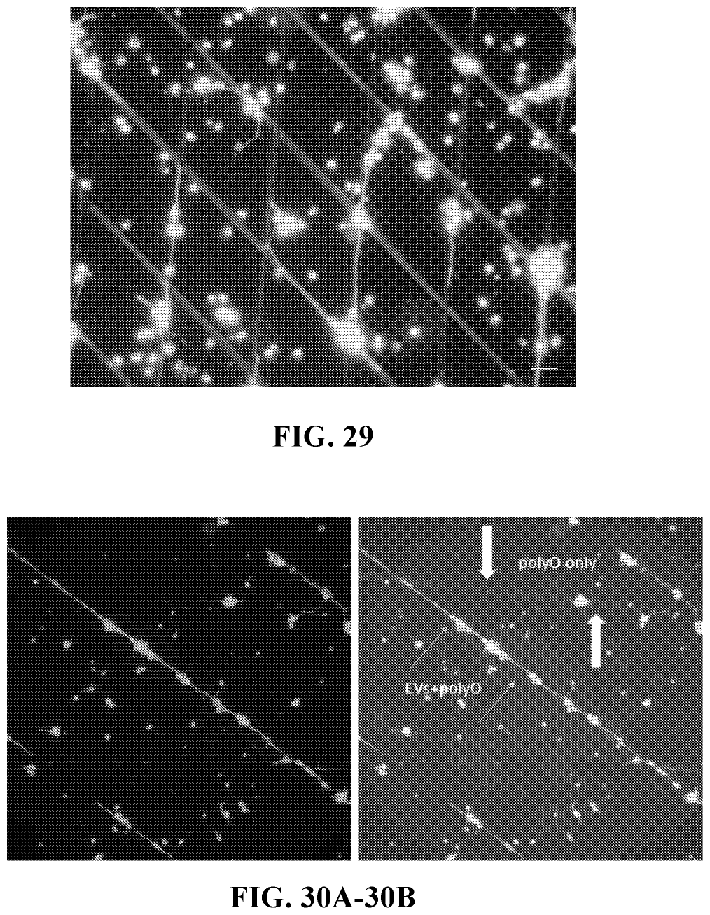

EVs can be stamped into patterns on surfaces (See e.g., FIGS. 28-29). EVs with a polyornithine (PolyO) were stamped into patterns onto a surface if a microslips and dried on the surface of the microslips. PolyO was patterned as a control. As shown in FIG. 30A-30B OSN derived EVs that were stamped into patterns and dried on a microslip were able to promote migration and extension of neurons along the patterned track. Neuron extension only occurred on EV containing patterned stripes.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A composition comprising: a population of olfactory sensory neuron (OSN) derived extracellular vesicles (EVs) enriched for EVs comprising and optionally expressing one or more cell adhesion molecules, wherein the one or more cell adhesion molecules are optionally one or more cadherins, one or more neural cell adhesion molecules (NCAMs), one or more epithelial cell adhesion molecule (EpCAM), one or more integrins, one or more plexins, or any combination thereof.

2. The composition of aspect 1, wherein the one or more cell adhesion molecules is/are, a delta protocadherin, optionally Pcdh1, Pcdh7, Pcdh8, Pcdh9, Pcdh10, Pcdh11, Pcdh17, Pcdh18, Pcdh19, Pcdh20 or any combination thereof.

3. The composition of any one of aspects 1-2, wherein the OSN derived EVs are microvesicles, exosomes, or a heterogenous population of both.

4. The composition of any one of aspects 1-3, wherein the OSN derived EVs comprise an exogenous cell adhesion molecule gene and/or gene product, wherein the exogenous cell adhesion molecule gene or gene product is optionally one or more cadherins, one or more neural cell adhesion molecules (NCAMs), one or more epithelial cell adhesion molecule (EpCAM), one or more integrins, one or more plexins, or any combination thereof.

5. The composition of aspect 4, wherein the OSN derived EVs comprise an exogenous Pcdh1, Pcdh7, Pcdh8, Pcdh9, Pcdh10, Pcdh11, Pcdh17, Pcdh18, Pcdh19, orPcdh20 gene or gene product, or any combination thereof.

6. The composition of any one of the aspects 1-5, further comprising a cargo, wherein the cargo is optionally a polynucleotide, a polypeptide, a nutrient (e.g., lipid, amino acid, carbohydrate, peptide, protein, sugar, vitamin, mineral, and/or the like), a genetic modifying system or component thereof, antibody or fragment thereof, aptamer, affibody, small molecule chemical agent (e.g., a therapeutic and/or prevention), an immunomodulator, a hormone, an antipyretic, an anxiolytic, an antipsychotic, an analgesic, an antispasmodic, an anti-epileptic, an anti-inflammatory agent, an anti-histamine, an anti-infective, a radiation sensitizer, a chemotherapeutic, a growth factor, a neurotransmitter agonist, a neurotransmitter antagonist, or any combination thereof.

7. The composition of any one of aspects 1-6, wherein the OSN derived EVs comprise one or more targeting moieties, wherein the targeting moiety is optionally a peptide, polypeptide, polynucleotide, sugar, a chemical molecule, a polymer, a lipid, a glycan, a peptidoglycan, or any combination or complex thereof (e.g., receptors, receptor ligands, antibodies and fragments thereof, aptamers, affibodies, antibody and/or aptamer epitopes, binding agents and their binding partners (e.g., biotin and streptavidin, enzymes and their substrates, a targeting nucleic acid, target nucleic acid and guided nuclease (e.g., miRNA, gRNA, RISC, Cas, etc.), guide nucleic acid for a guided nuclease system, and/or the like.

8. The composition of any one of aspects 1-7, wherein the composition is frozen, dehydrated, lyophilized, or otherwise modified for storage.

9. The composition of any one of aspects 1-8, wherein the composition is effective to stimulate axonal growth and/or increase the rate of axonal growth in a peripheral neuron, a central nervous system neuron, or both.

10. The composition of any one of aspects 1-9, wherein the composition is effective to increase correct axonal connectivity during neuron regeneration.

11. A formulation comprising: the composition of any one of the preceding claims; and a pharmaceutically acceptable carrier or excipient.

12. The formulation of aspect 11, wherein the formulation is adapted for oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, buccal, conjunctival, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradiscal, intraductal, intraduodenal, intradural, intraepidermal, intraesophageal, intragastric, intragingival, intraileal, intralesional, intraluminal, intralymphatic, intramedullary, intrameningeal, intraovarian, intrapericardial, intrapleural, intraprostatic, intrapulmonary, intrasinal, intraspinal, intrasynovial, intratendinous, intratesticular, subarachnoid, subconjunctival, subcutaneous, sublingual, submucosal, topical, transdermal, transmucosal, transplacental, transtracheal, transtympanic, or any combination thereof administration.

13. A method of treating a disease, disorder, and/or condition in a subject in need thereof, the method comprising: administering a composition or a formulation as in any one of the preceding aspects to the subject in need thereof.

14. A method of increasing/enhancing axonal growth and/or the rate of axonal growth during neuron development and/or regeneration, the method comprising: administering a composition or a formulation as in any one of the preceding aspects to the subject in need thereof.

15. A method of increasing neuron synapse formation, connectivity, or both during neuron development and/or regeneration, the method comprising: administering a composition or a formulation as in any one of preceding aspects to the subject in need thereof.

16. The method of any one of aspects 13-15, wherein the subject in need thereof has a nerve injury, nerve death, aberrant neuron connectivity, aberrant neuron activity, a neuropathy, or any combination thereof.

17. The method of any one of aspects 13-16, wherein the subject in need thereof has or is suspected of having a neurodegenerative disease, disorder, and/or condition.

18. The method of any one of aspects 13-17, wherein the subject in need thereof has, has had, or is suspected of having an epilepsy, a dementia (e.g., Dementia with Lewy Bodies, Vascular dementia, Frontotemporal Dementia, mixed dementia, Cruetzfeldt-Jakob disease), a stroke, Alzheimer's disease, Motor neuron disease, Huntington's disease, Parkinson's disease, a Parkinsonism (e.g., multiple system atrophy, corticobasal degeneration, diffuse Lewy body disease, spinal muscular atrophy, Friedreich ataxia, amyotrophic lateral sclerosis, and any combination thereof.

19. The method of any one of aspects 13-18, wherein the subject in need thereof has, has had, or is suspected of having a CNS neuron/nerve and/or a peripheral neuron/nerve injury, disease, disorder, and/or condition.

20. The method of any one of aspects 13-19, wherein the disease or disorder is a genetic disease, disorder, and/or condition.

21. The method of any one of aspects 13-19, wherein the disease or disorder is not a genetic disease, disorder, and/or condition.

22. A method of promoting stem cell division or differentiation and/or cell reprogramming, comprising: administering a composition or a formulation as in any one of the preceding aspects to a stem cell or epithelial cell or population thereof.

23. The method of aspect 22, wherein the cell is a differentiated cell.

24. The method of any one of aspects 22-23, wherein the cell is an epithelial cell.

25. The method of any one of aspects 22-24, wherein the cell is a neuron cell.

26. The method of any one of aspect 22, wherein the stem cell is an induced pluripotent stem cell.

27. A device comprising: a population of olfactory sensory neuron (OSN) derived extracellular vesicles (EVs), wherein the population of OSN derived EVs are fixed in a pattern on one or more surfaces on the device.

28. The device of aspect 27, wherein the population of OSN derived EVs are dried.

29. The device of any one of aspects 27-28, wherein the pattern is configured to direct correct neuron growth.

30. The device of any one of aspects 27-29, wherein the device is an implantable device.

31. A method of treating a nerve or neurodegenerative disease, disorder, and/or condition in a subject in need thereof, comprising: implanting the device as in any one of aspects 27-30 into the subject in need thereof.

32. A method of directing, increasing/enhancing axonal growth and/or the rate of axonal growth during neuron development and/or regeneration, the method comprising: implanting the device as in any one of aspects 27-31 into the subject in need thereof.

What is claimed is:

1. A composition comprising:
a population of olfactory sensory neuron (OSN) derived extracellular vesicles (EVs) enriched for EVs comprising and optionally expressing one or more cell adhesion molecules, wherein the one or more cell adhesion molecules are optionally one or more cadherins, one or more neural cell adhesion molecules (NCAMs), one or more epithelial cell adhesion molecule (EpCAM), one or more integrins, one or more plexins, or any combination thereof.

2. The composition of claim 1, wherein the one or more cell adhesion molecules is/are, a delta protocadherin, optionally Pcdh1, Pcdh7, Pcdh8, Pcdh9, Pcdh10, Pcdh11, Pcdh17, Pcdh18, Pcdh19, Pcdh20 or any combination thereof.

3. The composition of claim 1, wherein the OSN derived EVs are microvesicles, exosomes, or a heterogenous population of both.

4. The composition of claim 1, wherein the OSN derived EVs comprise an exogenous cell adhesion molecule gene and/or gene product, wherein the exogenous cell adhesion molecule gene or gene product is optionally one or more cadherins, one or more neural cell adhesion molecules (NCAMs), one or more epithelial cell adhesion molecule (EpCAM), one or more integrins, one or more plexins, or any combination thereof.

5. The composition of claim 4, wherein the OSN derived EVs comprise an exogenous Pcdh1, Pcdh7, Pcdh8, Pcdh9, Pcdh10, Pcdh11, Pcdh17, Pcdh18, Pcdh19, or Pcdh20 gene or gene product, or any combination thereof.

6. The composition of claim 1, further comprising a cargo, wherein the cargo is optionally a polynucleotide, a polypeptide, a nutrient, a genetic modifying system or component thereof, antibody or fragment thereof, aptamer, affibody, small molecule chemical agent, an immunomodulator, a hormone, an antipyretic, an anxiolytic, an antipsychotic, an analgesic, an antispasmodic, an anti-epileptic, an anti-inflammatory agent, an anti-histamine, an anti-infective, a radiation sensitizer, a chemotherapeutic, a growth factor, a neurotransmitter agonist, a neurotransmitter antagonist, or any combination thereof.

7. The composition of claim 1, wherein the OSN derived EVs comprise one or more targeting moieties, wherein the targeting moiety is optionally a peptide, polypeptide, polynucleotide, sugar, a chemical molecule, a polymer, a lipid, a glycan, a peptidoglycan, or any combination or complex thereof.

8. The composition of claim 1, wherein the composition is frozen, dehydrated, lyophilized, or otherwise modified for storage.

9. The composition of claim 1, wherein the composition is effective to stimulate axonal growth and/or increase the rate of axonal growth in a peripheral neuron, a central nervous system neuron, or both.

10. The composition of claim 1, wherein the composition is effective to increase correct axonal connectivity during neuron regeneration.

11. A formulation comprising:
the composition of claim 1; and
a pharmaceutically acceptable carrier or excipient.

12. The formulation of claim 11, wherein the formulation is adapted for oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, buccal, conjunctival, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradiscal, intraductal, intraduodenal, intradural, intraepidermal, intraesophageal, intragastric, intragingival, intraileal, intralesional, intraluminal, intralymphatic, intramedullary, intrameningeal, intraovarian, intrapericardial, intrapleural, intraprostatic, intrapulmonary, intrasinal, intraspinal, intrasynovial, intratendinous, intratesticular, subarachnoid, subconjunctival, subcutaneous, sublingual, submucosal, topical, transdermal, transmucosal, transplacental, transtracheal, transtympanic, or any combination thereof administration.

13. A method of treating a disease, disorder, and/or condition in a subject in need thereof, the method comprising:
administering a composition of claim 1 or a formulation thereof to the subject in need thereof.

14. The method of claim 13, wherein the subject in need thereof has a nerve injury, nerve death, aberrant neuron connectivity, aberrant neuron activity, a neuropathy, or any combination thereof.

15. The method of claim 13, wherein the subject in need thereof has or is suspected of having a neurodegenerative disease, disorder, and/or condition.

16. The method of claim 13, wherein the subject in need thereof has, has had, or is suspected of having an epilepsy, a dementia, a stroke, Alzheimer's disease, Motor neuron disease, Huntington's disease, Parkinson's disease, a Parkinsonism disease, corticobasal degeneration, diffuse Lewy body disease, spinal muscular atrophy, Friedreich ataxia, amyotrophic lateral sclerosis, or any combination thereof.

17. The method of claim 13, wherein the subject in need thereof has, has had, or is suspected of having a CNS neuron/nerve and/or a peripheral neuron/nerve injury, disease, disorder, and/or condition.

18. The method of claim 13, wherein the disease or disorder is a genetic disease, disorder, and/or condition.

19. The method of claim 13, wherein the disease or disorder is not a genetic disease, disorder, and/or condition.

20. A method of increasing/enhancing axonal growth and/or the rate of axonal growth during neuron development and/or regeneration, the method comprising:
administering a composition of claim 1 or a formulation thereof to the subject in need thereof.

21. The method of claim 20, wherein the subject in need thereof has a nerve injury, nerve death, aberrant neuron connectivity, aberrant neuron activity, a neuropathy, or any combination thereof.

22. The method of claim 20, wherein the subject in need thereof has or is suspected of having a neurodegenerative disease, disorder, and/or condition.

23. The method of claim 20, wherein the subject in need thereof has, has had, or is suspected of having an epilepsy, a dementia, a stroke, Alzheimer's disease, Motor neuron disease, Huntington's disease, Parkinson's disease, a Parkinsonism disease, corticobasal degeneration, diffuse Lewy body disease, spinal muscular atrophy, Friedreich ataxia, amyotrophic lateral sclerosis, or any combination thereof.

24. The method of claim 20, wherein the subject in need thereof has, has had, or is suspected of having a CNS neuron/nerve and/or a peripheral neuron/nerve injury, disease, disorder, and/or condition.

25. The method of claim 20, wherein the disease or disorder is a genetic disease, disorder, and/or condition.

26. The method of claim 20, wherein the disease or disorder is not a genetic disease, disorder, and/or condition.

27. A method of increasing neuron synapse formation, connectivity, or both during neuron development and/or regeneration, the method comprising:
administering a composition of claim 1 or a formulation thereof to the subject in need thereof.

28. The method of claim 27, wherein the subject in need thereof has a nerve injury, nerve death, aberrant neuron connectivity, aberrant neuron activity, a neuropathy, or any combination thereof.

29. The method of claim 27, wherein the subject in need thereof has or is suspected of having a neurodegenerative disease, disorder, and/or condition.

30. The method of claim 27, wherein the subject in need thereof has, has had, or is suspected of having an epilepsy, a dementia, a stroke, Alzheimer's disease, Motor neuron disease, Huntington's disease, Parkinson's disease, a Parkinsonism disease, corticobasal degeneration, diffuse Lewy body disease, spinal muscular atrophy, Friedreich ataxia, amyotrophic lateral sclerosis, or any combination thereof.

31. The method of claim 27, wherein the subject in need thereof has, has had, or is suspected of having a CNS neuron/nerve and/or a peripheral neuron/nerve injury, disease, disorder, and/or condition.

32. The method of claim 27, wherein the disease or disorder is a genetic disease, disorder, and/or condition.

33. The method of claim 27, wherein the disease or disorder is not a genetic disease, disorder, and/or condition.

34. A method of promoting stem cell division or differentiation and/or cell reprogramming, comprising:
administering a composition of claim 1 or a formulation thereof to a stem cell or epithelial cell or population thereof.

35. The method of claim 34, wherein the cell is a differentiated cell.

36. The method of claim 34, wherein the cell is an epithelial cell.

37. The method of claim 34, wherein the cell is a neuron cell.

38. The method of claim 34, wherein the stem cell is an induced pluripotent stem cell.

39. A device comprising:
a population of olfactory sensory neuron (OSN) derived extracellular vesicles (EVs), wherein the population of OSN derived EVs are fixed in a pattern on one or more surfaces on the device.

40. The device of claim 39, wherein the population of OSN derived EVs are dried.

41. The device of claim 39, wherein the pattern is configured to direct correct neuron growth.

42. The device of claim 39, wherein the device is an implantable device.

43. A method of treating a nerve or neurodegenerative injury, disease, disorder, and/or condition in a subject in need thereof, comprising:
implanting the device of any one of claims 39-42 into the subject in need thereof.

44. A method of directing, increasing/enhancing axonal growth and/or the rate of axonal growth during neuron development and/or regeneration, the method comprising:
implanting the device of any one of claims 39-42 into the subject in need thereof.
